(19) United States
(12) Patent Application Publication
(10) Pub. No.: US 2011/0117156 A1
(43) Pub. Date: May 19, 2011

(54) COMPOSITIONS AND ARTICLES CONTAINING AN ACTIVE LIQUID IN A POLYMERIC MATRIX AND METHODS OF MAKING AND USING THE SAME

(75) Inventors: Geng Lin, Savannah, GA (US); Mark S. Pavlin, Kingsport, TN (US)

(73) Assignee: ARIZONA CHEMICAL COMPANY, Jacksonville, FL (US)

(21) Appl. No.: 12/951,421

(22) Filed: Nov. 22, 2010

Related U.S. Application Data
(63) Continuation-in-part of application No. 11/945,184, filed on Nov. 26, 2007.
Application No. 11/945,184 is a continuation-in-part of application No. 11/140,160, filed on May 27, 2005, now abandoned.
(60) Provisional application No. 60/870,822, filed on Dec. 19, 2006.
(60) Provisional application No. 60/574,759, filed on May 27, 2004.
(60) Provisional application No. 60/618,449, filed on Oct. 13, 2004.

(51) Int. Cl. A01N 25/10; A01N 35/04; C11D 3/60; A01N 53/08; A01N 37/18; A01P 7/04; A01P 19/00; A61L 9/01; C10M 107/40

(52) U.S. Cl. 424/409; 514/693; 510/516; 510/515; 514/531; 514/617; 424/76.1; 508/551

(57) ABSTRACT

Described herein are compositions and articles containing a polymeric matrix and an active liquid intermixed with at least a portion of the polymeric matrix. Methods of making and using the compositions and articles are also described herein.

### CROSS-REFERENCE TO RELATED APPLICATIONS

This application is a continuation-in-part of U.S. patent application Ser. No. 11/945,184, filed Nov. 26, 2007, which claims priority to U.S. Provisional Patent Application No. 60/870,822, filed Dec. 19, 2006, and is a continuation-in-part of U.S. patent application Ser. No. 11/140,160, filed May 27, 2005, which claims priority to U.S. Provisional Patent Application Nos. 60/574,759, filed May 27, 2004, and 60/618,449, filed Oct. 13, 2004, all of which are incorporated herein by reference in their entireties.

### FIELD

Compositions and articles containing an active liquid intermixed with a polymer matrix, as well as methods of making and using the same, are described herein.

### BACKGROUND

The curing and/or cross-linking of polymeric systems, for example epoxy systems, is described in textbooks and industrial handbooks such as “Handbook of Epoxy Resins” by Henry Lee and Kris Neville (McGraw Hill, 1967), “The Epoxy Formulators Manual” by the Society of Plastics Industry, Inc. (1984), and the Encyclopedia of Science and Technology (Kirk-Othmer,

John Wiley & Sons, 1994). Until recently, curing such systems and others related thereto in a manner capable of immobilizing active liquids, such as those having and/or containing fragrance, has been very difficult, especially when durability and performance under a dynamic range of operation conditions are required from such systems.

For example, JP 032558899A requires the use of a solid powder system, while JP07145299 requires the use of a pre-formed urethane-containing epoxy resin cross-linked in the absence of a polyamine and/or an active liquid containing a perfume. Further, the above-mentioned JP references refer specifically and only to fragranced articles, such as air fresheners. Because of this narrow goal to make such articles, the reaction and reaction products described therein fail to have a dynamic range of performance capabilities. Moreover, they fail to provide a product that is durable in the absence of a support. Therefore, a need arises for controllable reaction conditions that yield dynamic reaction products containing durable matrices capable of immobilizing any and/or all types of active liquids therein.

Compositions such as fragrance objects, even more specifically air fresheners, are well known devices that release a fragrance into the air of a room of a house, area of a public building (e.g., a lavatory), or the interior of a car to render the air in that area more pleasing to the occupant. Only substantially non-aqueous gels, for example, the thermoplastic polyamide-based products described in U.S. Pat. Nos. 6,111,655 and 6,503,577 and the thermo-set poly(amide-acid)s of U.S. Pat. No. 5,780,527 and U.S. Pat. No. 6,846,491, are homogeneous, transparent solids that can be easily charged, when in liquid form, to a mold and thus made into a visually attractive solid shape without the use of a means of support. However, during preparation of thermoplastic gels, the components must be heated to a temperature above the gelation temperature of the mixture, a process detrimental to the volatile and sometimes temperature sensitive active liquid such as fragrance, pesticide, or surfactant. During storage or use, these gels must not be exposed to low temperatures because they can turn unattractively cloudy. Furthermore, these gels must not be exposed to high temperatures because they will turn liquid, losing their shape or leaking from their container. These drawbacks are serious for air fresheners necessarily exposed to a dynamic range of temperatures, such as car interior fresheners. The latter are often exposed to low temperatures in winter and temperatures in excess of 110° F. on summer days when the car is parked in direct sunlight. In addition, thermoplastic gels are soft solids that are easily deformed if scraped, dropped, poked, or wiped. Thus, these conventional gels do not provide compositions and/or articles that are readily durable and capable of operating at a wide range of operating parameters.

Air care articles can contain a variety of fragrances. Aldehydes are common fragrance oil ingredients and can react with primary amines and interfere with polymer matrix setting times, especially for polymer matrices based on isocyanate-polyamine curing systems. It is not unusual that compositions fail to form the desired articles because aldehydes consume free primary amine groups.

(Polymer-)-NH₂+O═C(R)—H⇄(Polymer-)-N═C(R)H+H₂O

This drawback significantly limits a consumer's options to choose fragrances and makes product manufacture very difficult.

### SUMMARY

Described herein are compositions and articles containing an active liquid intermixed with a polymeric matrix and methods of making and using the same. The compositions include a polymeric matrix comprising the reaction product of a polyamine and a compound having at least two functional groups and an active liquid intermixed with at least a portion of the polymeric matrix. The functional groups are selected from the group consisting of epoxy groups, isocyanate groups, anhydride groups, and acrylate groups. The polyamine and the compound are reacted in the presence of the active liquid. In some examples, the polyamine is a polyamide polyamine and/or a secondary amine terminated polyamine. The reactive amine groups of the polyamine can include amino groups derived from at least one of ortho-aminobenzoic acid or para-aminobenzoic acid. In some embodiments, the polyamine is a non-water soluble polyamide polyamine with a molecular weight in the range of 4,000 to 30,000 Daltons.

In some examples, the active liquid is present in an amount from 10 weight % to 85 weight % based on the weight of the composition (e.g., from 50 weight % to 85 weight % based on the weight of the composition). The active liquid can include, for example, a therapeutic active liquid, a nutraceutical active liquid, a cosmeceutical active liquid, a pesticidal active liquid, a laundry care active liquid, a fragrance, or a mixture thereof. In some examples, the compound includes at least one non-aromatic isocyanate compound.

The composition described herein can be in the form of a gel. In other examples, the composition can be in the form of a particle and can be present in an aqueous dispersion. The particle size can be, for example, from 1 micron to 100 microns (e.g., from 2 microns to 15 microns). Also described herein are articles comprising a porous support material and the composition described herein.

Methods of preparing the compositions are also provided herein. The methods can include reacting a polyamine with a compound having at least two functional groups, the functional groups selected from the group consisting of epoxy groups, isocyanate groups, anhydride groups, and acrylate groups in the presence of an active liquid. In some examples, the polyamine is liquid at room temperature. In some examples, the polyamine has an amine number of from 10 meq KOH/g to 100 meq KOH/g. The polyamine can have a viscosity of 500 cP or less at 150° C. In some examples, the reacting step occurs at room temperature.

The details of one or more embodiments are set forth in the described below. Other features, objects, and advantages will be apparent from the description and the claims.

### DETAILED DESCRIPTION

Compositions and/or articles containing an active liquid-intermixed polymeric matrix and methods for their preparation and use are described herein. The polymeric matrix can be a thermoset (i.e., a cross-linked) polymeric matrix that includes an active liquid intermixed within the matrix. In some embodiments, the active liquid is uniformly (i.e., homogenously) intermixed within the matrix. The polymeric matrices described herein are durable and stable over a wide range of conditions.

The polymeric matrix is the reaction product of a polyamine and a compound having at least two functional groups selected from the group consisting of epoxy groups, isocyanate groups, anhydride groups, and acrylate groups. The polyamine can include a polyamide polyamine (PAPA) and/or a secondary amine terminated polyamine (SATPA). The reaction is carried out in the presence of the active liquid. A small amount of water can be intermixed as a part of the active liquid. In some examples, the composition can then be dispersed in an aqueous phase in the form of a particle dispersion.

The compound having at least two functional groups selected from the group consisting of epoxy groups, isocyanate groups, anhydride groups, and acrylate groups can be, for example, crosslinking agents. In some examples, the crosslinking agent is an epoxy crosslinking agent (i.e., a compound having at least two functional groups that include an epoxy group). The epoxy crosslinking agent can be any epoxy. In some examples, the epoxy crosslinking agent is in the form of a liquid. Examples of liquid epoxy resins that can be used in the compositions described herein include diglycidyl ethers of bisphenol A and F, commercially available as EPON 828 and EPON 8620 from Resolution Performance Products (Houston, Tex.); hydrogenated glycidyl ethers of bisphenol A, commercially available as EPALLOY 5000 and EPALLOY 5001 from CVC Specialty Chemicals; Moorestown, N.J.; and diglycidyl ethers of butanediol, cyclohexane dimethanol, neopentyl glycol, dimer acid, and trimethylolpropane, all commercially available from Resolution Performance Products in the HELOXY Modifier product line. Further examples of the epoxy containing compound described herein can be found in “Handbook of Epoxy Resins” by Henry Lee and Kris Neville (McGraw Hill, 1967), “The Epoxy Formulators Manual” by the Society of Plastics Industry, Inc. (1984), and the Encyclopedia of Science and Technology (Kirk-Othmer, John Wiley & Sons, 1994). The above-mentioned epoxy-containing compounds are merely representative and many additional epoxy-containing compounds are applicable.

In some examples, the compound having at least two functional groups described herein can be a compound including at least two anhydride functional groups (i.e., a polyanhydride). In some examples, the polyanhydride is in the form of a liquid. For example, the anhydride can be a solid polymer dissolved in a suitable carrier liquid. In some examples, the polyanhydride is not a maleated polyolefin rubber. Examples of polymers for the anhydrides include, for example, maleated olefin polymers other than a maleated rubber (e.g., a polybutadiene or a poly(isobutylene)), olefin-maleic anhydride co-polymers, and alpha-olefin-maleic anhydride alternating co-polymers. Specific examples of suitable anhydride-functional polymers include styrene-maleic anhydride copolymers such as DYLARK 232 and DYLARK 332, available from NOVA Chemicals (Moon Township, Pa.), and poly(l-octadecene-alt-maleic anhydride), commercially available from Chevron Corporation (San Ramon, Calif.). These anhydride-containing polymers are representative and many additional anhydride-containing polymers are applicable.

In some examples, the compound having at least two functional groups described herein can be a compound including at least two isocyanate functional groups (i.e., a polyisocyanate). In some examples, the polyisocyanate is in the form of a liquid. In some examples, the compound includes at least one non-aromatic isocyanate compound. Specific examples of the isocyanate-containing compounds include aliphatic difunctional isocyanate materials such as liquid diisocyanates (e.g., isophorone diisocyanate and bis(4-isocyanato cyclohexyl) methane). The polyfunctional isocyanates can have low volatility and reduced toxicity. Examples of these isocyanates include the DESMODUR N-series aliphatic isocyanurates (e.g., DESMODUR N-3300, DESMODUR N-3600, and DESMODUR N-3800), and the DESMODUR Z-series (e.g., DESMODUR Z4470), all commercially available from Bayer Corporation, Industrial Chemicals Division (Pittsburgh, PA). These isocyanate-containing compounds are representative and additional isocyanate-containing compounds are applicable. In some embodiments, the equivalent weight for the isocyanate-containing compounds is in the range of 180 to 500.

As discussed below, certain functional groups react with certain polyamines faster than other functional groups. For example, the isocyanate functional group reacts with an amine functional group significantly faster than does the epoxy functional group so that polyamine compounds suitable for the cross-linking reaction with isocyanates are not necessarily satisfactory for use with epoxies. A polyamine compound for reaction with epoxy-functional compounds can be a liquid at room temperature (e.g., 25° C.); can dissolve in, and can be compatible with, many active liquids; can have a viscosity, measured at 100° C., of no greater than about 100 cP; and can have an amine number of from 100 to 1200 meq KOH/g. For example, the amine number can be 100, 200, 500, 750, 1000 and 1200 meq KOH/g, including any and all ranges and subranges there between. Suitable polyamines include, for example, 1,2-diaminocyclohexane, isophorone diamine, meta-xylene diamine, and 1,3-bis(aminomethyl)cyclohexane (1,3-BAC). In some examples, the polyamines can be poly(alkyleneoxy) polyamines (i.e., polyether amines) that are liquid at 25° C. and include polyether segments such that greater than 50% by weight of the amine is derived from a polyether. For example, the polyether can be an oligomerized ethylene oxide, propylene oxide, butylenes oxide, tetrahydrofuran, or combinations of these supplied by, for example, Huntsman Corporation (The Woodlands, Tex.) and BASF Corporation (Florham Park, N.J.). Examples of suitable polyamines include, for example, JEFFAMINE D-230, D-400, D-2000, T-5000, T-403, and XT J511 XTJ-511, all polyether diamines commercially available from Huntsman Corporation (The Woodlands, Tex.). Liquid polyamines can also be chosen from the polyamido-amine family, examples of which are the UNIREZ series of amidoamide-amine curing agents commercially available from Arizona Chemical (Jacksonville, Fla.). These materials are known to impart adhesion and have lowered skin sensitivity. In some examples, the amines can be mixtures of two or more amines blended to optimize viscosity, reaction rate and product performance.

In some examples, the polyamine compound suitable for reaction with isocyanate-functional compounds can be a material having a polymeric backbone comprising repeating monomer units terminated by amine groups that are different from the repeating amine groups. This polymeric polyamine can be a liquid at a temperature below 50° C., e.g., a liquid or low melt point amine. For example, the polyamine can be a liquid at normal room temperature. In some examples, the amine has a melting or softening point at or below 50° C., (e.g., 45° C., 40° C., 30° C., 20° C., and 10° C., including any and all ranges and subranges there between). In some examples, the polyamine is a liquid and/or tacky and/or a semisolid at a temperature below 10° C.

Further, in some examples, the polymeric polyamine dissolves in, and is compatible with, many active liquids; has a number-average molecular weight of greater than 1,000; has an amine number of from 10 to 100 meq KOH/g; and has a viscosity, measured at 150° C., of no greater than about 500 cP. The amine number can be, for example, 10, 25, 50, 75, or 100 meq KOH/g, including any and all ranges and subranges there between. Further, the viscosity, measured at 150° C., of the polyamine can be 500 cP or less. For example, the viscosity, measured at 150° C., of the polyamine can be about 450 cP, 350 cP, 250 cP, 150 cP, and 100 cP, including any and all ranges and subranges there between.

In some examples, the polymeric polyamine for reacting with isocyanate-functional compounds can be a polyamide polyamine (or “PAPA”). The polyamide polyamines can be polyamide polyether block copolymers resulting from the reaction of one or more polyalkyleneoxy polyamines with one or more aliphatic polyacids as further described below. Such ether-based polyamide polyamines can be made by reacting a polyacid or mixture of polyacids with a stoichiometric excess of polyether polyamine admixed with optional lower diamines including piperazine, ethylene diamine, isophorone diamine, hexamethylene diamine, 2-methyl-1,5-pentane diamine, and the like. Suitable polyacids for the preparation of PAPAs are adipic acid, azeleic acid, sebacic acid, dodecandioic acid or other aliphatic diacid or its ester equivalent. Use of such diacids and a majority amount of poly(alkyleneoxy) polyamine, determined as >50% of all amine equivalents present, ensures that the resulting polyamide will have good solubility in a wide range of liquids including in certain cases, water. In some examples, the polyamide polyamine is not soluble in water. The amine number of the PAPA can be less than 100, as measured by titration with dilute alcoholic hydrochloric acid and expressed as mg KOH/g sample. In some examples, the amine number of the PAPA is less than 80 mg KOH/g or less than 70 mg KOH/g.

Examples of suitable PAPAs include the reaction products of polymerized fatty acids, also known as dimer acids (e.g., material produced by Arizona Chemical Company under the trade name “UNIDYME”; Unichema Corporation (Wilmington, Del.) under the name “PRIPOL”; and Cognis Corporation (Cincinnati, Ohio) under the trade name “EMPOL”) and a stoichiometric excess of one or more poly(alkyleneoxy) polyamines chosen from the group of Huntsman JEFFAMINE polyamines, including, for example, D-400, D-2000, T-403, and XTJ-500. In these examples, the resulting polymeric polyamines can be liquid at room temperature, have an acid value of less than about 5 and an amine value of from about 10 to about 70; and have a viscosity of less than 500 cP measured at 150° C. In some examples, the PAPA is liquid at room temperature, has an acid value of less than 2 and an amine value of 20-60, and has a viscosity of less than 300 cP at 150° C. For example, a polymeric polyamine can be obtained by reacting 29.5 weight % of PRIPOL 1009 hydrogenated dimer acid, 44.5 weight % of JEFFAMINE D-2000, 22.5 weight % of JEFFAMINE® D-400, and 3.5 weight % of JEFFAMINE T-403 at 215° C. under a sweep of dry nitrogen until the acid number drops to about 1.0 and the amine value is adjusted to be about 30-40. The resulting material can be, for example, a viscous liquid at room temperature with a viscosity of about 100 cP at 130° C. and a weight average molecular weight of about 25,000 Daltons.

Reaction rates for forming the matrix vary with the type of terminal amine present in the polymeric polyamine component. The shortest cure times result from the use of a compound whose polymer chain terminates in an aliphatic primary or secondary amine. Amines hindered by substitution with a bulky group such as a tertiary butyl moiety react more slowly. The longest cure times result from the use of a polymeric polyamine terminated with a certain type of aromatic amine whose aromatic ring bears a carbonyl, particularly an ester or amide group, or other strong electron-withdrawing group. While it is believed that the carbonyl-substituted aromatic amines can be utilized for reaction with any of the functional groups described herein, they are particularly useful when the functional group is the highly-reactive isocyanate group.

While any such terminal carbonyl-substituted aromatic amine can be used, non-limiting examples of polyamines are those derived from para-aminobenzoic acid and ortho-aminobenzoic acid. These compounds are readily incorporated onto the termini of polyamides described herein by reaction with the specified polyamines along with the specified diacids. A PAPA can include, for example, a polymer produced by reacting any of the above-described diacids and ether diamines in the presence of para-amino benzoic acid and/or ortho-amino benzoic acid. For example, a PAPA can be obtained by reacting 24.0 weight % PRIPOL 1009 hydrogenated dimer acid, 5.0 weight % para-aminobenzoic acid, 54.0 weight % JEFFAMINE D-2000, 11.5 weight % JEFFAMINE D-400, and 5.5 weight % JEFFAMINE® T-403 at 215° C. under a sweep of dry nitrogen until the acid number drops to about 1.0 and the amine value is adjusted to 15 by non-potentiometric titration and 30-35 by potentiometric titration. This material is a viscous liquid at room temperature with a viscosity of about 250 cP at 130° C. and a weight average molecular weight of about 13,000 Daltons.

In some examples, the weight-average molecular weight (Mw) and/or number-average molecular weight (Mn) of the PAPA can be as high as desired but can be limited by the desired amine value and viscosity. For example, the Mw can be in the range of 3000-40,000 Daltons and can be greater than 3000, 4000, 5000, 6000, 7000, 8000, 9000, or 10,000 and/or less than 40,000, 38,000, 36,000, 34,000, 32,000 or 30,000 Daltons. Accordingly, the polydispersity can be any value but is desirably greater than 1.5 and less than 6, or in the range 2.0-4.0, including any and all ranges and subranges there between.

Co-diacids and co-diamines can be used to prepare PAPAs described herein in an amount of less than 50% on an equivalents basis. Co-diacids can be, for example, adipic acid and similar linear aliphatic diacids. Co-diamines can include, for example, ethylene diamine, piperazine, 1,2-diaminocyclohexane, isophorone diamine, 1,3-bis(aminomethyl)cyclohexane, dimer diamine (e.g., VERSAMINE 551, commercially available from Cognis Corporation (Cincinnati, Ohio)), hexamethylene diamine, 2-methyl-1,5-pentane diamine, and similar linear, branched and cyclic aliphatic diamines. The polyamidification reaction can be carried out in the presence of catalysts known to increase the reaction rate such as acids, particularly para-toluene sulfonic, phosphoric and sulfuric acids, and with removal of water of reaction via application of a vacuum.

Suitable PAPAs further include those that are not liquid at room temperature. In some examples, the non-liquid PAPAs can be solid at room temperature (e.g., low melting polyamines). These PAPAs can result from the reaction of a major diacid portion of 1,4-cyclohexane dicarboxylic acid and a stoichiometric excess of polyamine, the majority of which is a poly(alkyleneoxy) polyamine chosen from the group of Huntsman JEFFAMINE® polyamines, including, for example, D-400, D-2000, T-403, and XTJ-500 such that, after the reaction is complete, the PAPA is a solid at 25° C., has an acid value of less than 5, has an amine value of from about 10 to about 70, and has a Ring & Ball softening point less than 50° C. For these polyamides, the dimer acid can be used as a co-diacid along with other co-diacids such as those mentioned above. Co-diamines can also be used to prepare the PAPAs described herein.

Further examples of polymeric polyamines for use in the compositions and articles described herein include those described in U.S. Pat. Nos. 6,399,713, 6,870,011; and 6,956,099, which are incorporated, in their entireties, herein by reference.

In some examples, the polyamine is a secondary amine terminated polyamine (SATPA). The amine number of the SATPA can be 100 meq KOH/g or less. For example, the SATPA can have an amine number from 10 to 100 meq KOH/g. The composition can be, for example, a reaction product of a secondary amine terminated polyamine (SATPA) and an isocyanate cross-linking agent in the presence of an active liquid to be intermixed. The gel composition can be prepared by blending the SATPA, the liquid actives, and the cross-linking agent.

The form of the composition can depend on the reactants used to form the polymeric matrix. For example, the polymeric matrix can include the reaction product of a secondary amine terminated polyamine and a compound having at least two functional groups selected from the group consisting of epoxy groups, isocyanate groups, anhydride groups, and acrylate groups. In some embodiments, the polymeric matrix can include the reaction product of a secondary amine terminated polyamine and a compound having at least two isocyanate functional groups. In some examples, the compositions can be in the form of a gel (e.g., a clear, crosslinked polymeric gel). In other examples, the polymeric matrix can include the reaction product of a polyamide polyamine and the compound having the at least two functional groups as described above. In these examples, the resulting compositions can be in the form of a particle (e.g., a particle in an aqueous dispersion). The particle size of the particles can be from 1 micron to 100 microns, for example, from 2 microns to 15 microns. For example, the particle size of the particles can be 3 microns, 4 microns, 5 microns, 6 microns, 7 microns, 8 microns, 9 microns, 10 microns, 11 microns, 12 microns, 13 microns, or 14 microns.

As described above, the reaction to produce the polymeric matrix is carried out in the presence of an active liquid. The resulting polymeric matrix includes the active liquid intermixed with at least a portion of the matrix and, in some embodiments, throughout the matrix. The active liquid can be any liquid that imparts a function upon the resultant composition and/or article a function. For example, the active liquid can be a volatile or non-volatile organic liquid. In some examples, the active liquid can be a semi-solid or a solid dissolved in a carrier liquid (e.g., a diluent). Examples of suitable active liquids include therapeutic active liquids, nutraceutical active liquids, cosmeceutical active liquids, pesticidal active liquids, laundry care active liquids, fragrance oils, surface treating chemicals, radio-tracers, surfactants, or a mixture of these.

In some examples, the active liquid can be a fragrance oil (i.e., a scent or perfume). A fragrance oil can be any blend of the large number of synthetic aroma chemicals and aromatic natural oils known to one of skill in the art. Examples of useful classes of chemicals include esters such as linalool acetate and butyl acetate (present in banana oil), phenols such as methyl salicylate (present in oil of wintergreen), ethers such as 1,8-cineole (present in eucalyptus oil), alcohols such as geraniol (present in rose oil), ketones such as menthone (present in spearmint oil), and aldehydes such as cinnamaldehyde (present in cinnamon oil). Further examples of suitable aldehydes include citral, benzaldehyde, p-alkyl-substituted benzaldehydes, anisaldehyde, vanillin, heliotropin, and alkyl-substituted cinnamic aldehydes.

In some situations, aldehydes may react with primary amines and interfere with polymer matrix setting times, especially for polymer matrices based on isocyanate-polyamine curing systems. While not wishing to be limited to theory, it is believed that secondary amines do not react with aldehydes because they do not have a proton available. Thus, the aldehydes in fragrances do not interfere with secondary amine crosslinking agent in preparation of the compositions and articles described herein. The interference from aldehydes in fragrances can be eliminated, which also leads to more consistent products and efficient manufacturing. Additionally, a number of fragrance types can be used to prepare the intermixed fragrance oils with high fragrance loading (e.g., greater than 50%, greater than 55%, greater than 60%, greater than 65%, greater than 70%, greater than 75%, greater than 80%, or greater than 85%).

Specific examples of the many hundreds of commercially available fragrance oils useful for the compositions described herein are Ocean, Country Wildflower, Spring Meadow, and Morning Rain, supplied by Continental Aromatics (Hawthorne, N.J.); MacIntosh supplied by Orlandi, Inc. (Farmingdale, N.Y.); Evergreen, Green Apple, and Yankee Home supplied by Belle Aire Fragrances (Mundelein, Ill.); Cherry, Vanilla, Downey, and Mulberry supplied by Aromatic Fragrances and Flavors International (Marietta, Ga.); Garnet supplied by International Fragrances Technology, Inc. (Canton, Ga.); Crisp Breeze, Tropical Fragrance, and Oceanside Mist supplied by Atlas Products (Tinley Park, Ill.); and Orange Twist, Linen Fresh, and Country Garden supplied by Wessel Fragrances (Englewood Cliffs, N.J.).

The active liquid can be used at a level so as to impart efficacy to the composition for the intended application. The active ingredient can be extremely potent and need be present only in a very low level, e.g., less than 0.1%. In such a case, the active liquid is said to be the solution of potent agent in carrier. In these examples, the active liquid (or potent agent dissolved in carrier) can be used in the compositions and/or articles at levels from 1% for lightly-loaded objects to 90% or more. The loading can depend on the function of the particular active liquid, polymer matrix, and any other compounds present. It can also depend upon the final configuration of the formed product, that is, whether it is free-standing, contained, or supported. In some examples, the active liquid can be present in an amount of from 10 weight % to 85 weight % or from 50 weight % to 85 weight %. For example, the amount of active liquid can be 1%, 2%, 5%, 10%, 15%, 20%, 30%, 40%, 50%, 60%, 70%, 80%, or 90% inclusive of all ranges and subranges there between.

In some examples, the fragrance oil level for air fresheners can be from 15-75% (e.g., from 30-70%) by weight of the finished article not counting the weight of any embedded objects. The amount of fragrance oil can be 15%, 20%, 25%, 30%, 40%, 50%, 60% or 75% by weight of the composition (not counting the weight of any supports or embedded objects) inclusive of all ranges and subranges there between. Inactive diluent or plasticizer can be present in an additional amount such that the total liquid level can be from 20% to 90% by weight of the composition, for example, from 40% to 80% by weight of the composition.

Similarly, the mixture of reactive components, active liquid and optional liquids, while still uncured, can be dispersed in water or other aqueous medium and the resulting oil-in-water emulsion stabilized by means of a surfactant. Droplets of inventive composition thus emulsified cure to form a dispersion of solid immobilized active liquid particles in water. The surfactant can be anionic, cationic, or non-ionic in nature. Examples include the anionic salt sodium lauryl sulfate, the cationic quaternary ammonium salts di(hydrogenated tallow) dimethyl ammonium chloride, cocamido propyl betaine, and dibenzyl dimethyl ammonium chloride, and the non-ionic polyethoxylated sorbitan mono-oleate. Such an emulsion is a milky liquid and can, as such, be impregnated into a porous medium such as paper, cardboard, cellulose pad, cellulose pulp, felt, fabric, a porous synthetic foam, a porous ceramic, activated carbon, soil, diatomaceous earth, kieselguhr, charcoal, silica, clay, and the like or coated onto a non-porous substrate included but not limited to plastic films, metallic foils, rubber, ceramics, wood, glass, and leather.

Surfactant compounds can themselves be active compounds when used in excess of the amount needed to stabilize the gel dispersion. The surfactants can be used with or without water. Surfactants thus intermixed within the polymeric matrix are released slowly into their use environment along with the fragrance and other active components, and can thus serve as, for example, a toilet air freshener/cleaner, a pesticide/disinfectant, or a fabric softener in a laundry dryer either in the form of a liquid or, if impregnated into a porous medium, a sheet.

In some examples, the active liquid can be a liquid pesticide or a solid pesticide dissolved in a carrier liquid. As used herein, pesticide refers to any substance or mixture of substances intended for preventing, destroying, repelling, or mitigating any organism that causes or is able to cause harm or annoyance to humans, valuable animals (e.g., livestock), or valuable plants (e.g., flowers, trees, and food crops). Pesticides include chemical substances or biological agents (such as viruses or bacteria) used to control insects, plant pathogens, weeds, mollusks, birds, mammals, fish, nematodes (roundworms) and microbes that compete with humans for food, destroy property, spread disease, or are a nuisance. Because many pesticides are poisonous to humans, it is useful to control their application and release by, for example, dissolving them in a harmless carrier liquid and then intermixing and immobilizing them within the polymeric matrix. The pesticides can be naturally derived or synthetic. Examples of synthetic pesticides include organophosphates, carbamates, organochlorines, and pyrethroids.

Organophosphates and carbamates can affect the nervous system by disrupting the enzyme that regulates acetylcholine, a neurotransmitter. They usually are not persistent in the environment. Immobilization by intermixing, then, can help the organophosphates to be effective for a longer period of time without harming the environment. Organochlorines (e.g., DDT and chlordane) were commonly used in the past, but many have been removed from the market due to their health and environmental effects and their persistence. Pyrethroids were developed as synthetic versions of the naturally occurring pyrethrin to increase stability in the environment and lower their cost.

Some pesticides are derived from such natural materials as animals, plants, bacteria, an example being the naturally-occurring material, pyrethrin, extracted from chrysanthemums. Biopesticides include microbial pesticides that consist of a microorganism (e.g., a bacterium, fungus, virus or protozoan) as the active ingredient. Microbial pesticides can control many different kinds of pests, although each separate active ingredient is relatively specific for its target pest. For example, there are fungi that control certain weeds, and other fungi that kill specific insects. The most widely used microbial pesticides are subspecies and strains of Bacillus thuringiensis, or Bt.

Pesticides can be classified according to the type of pest that they combat. Examples of useful pesticides include algicides that control algae in lakes, canals, swimming pools, water tanks, and other sites; antifouling agents that kill or repel organisms that attach to underwater surfaces, such as boat bottoms; antimicrobials that kill microorganisms (such as bacteria and viruses); attractants that attract pests (for example, to lure an insect or rodent to a trap) including foods such as sugar; biopesticides that are active agents derived from natural materials such as animals, plants, bacteria, and certain minerals; biocides that kill microorganisms, disinfectants and sanitizers that kill or inactivate disease-producing microorganisms on inanimate objects, fungicides that kill fungi (including blights, mildews, molds, and rusts); herbicides that kill weeds and other plants that grow where they are not wanted; insecticides that kill insects and other arthropods, miticides (also called acaricides) that kill mites that feed on plants and animals; microbial pesticides that kill, inhibit, or outcompete pests, including insects or other microorganisms; molluscicides that kill snails and slugs; nematicides that kill nematodes (microscopic, worm-like organisms that feed on plant roots); ovicides that kill eggs of insects and mites; pheromones that disrupt the mating behavior of insects; repellents that are chemicals that repel pests, including insects (such as mosquitoes) and birds from a surface such as skin or seeds; rodenticides that sicken, repel, or kill mice and other rodents; insect growth regulators that disrupt the molting, maturity from pupal stage to adult, or other life processes of insects; and plant growth regulators that are substances (excluding fertilizers or other plant nutrients) that alter the expected growth, flowering, or reproduction rate of plants.

Without meaning to be exhaustive, specific examples of pesticides that can be used as the active liquid include: 2,4-D, 2,4-DB, DCPA (chlorthal), MCPA, abamectin, acephate (orthene), acetochlor, acifluorfen, alachlor, aldicarb, allethrin, ametryn, amitraz, atrazine, azadirachtin, azinophos-methyl, Bacillus Thuringiensis, bendiocarb, benomyl, bensulide, bentazon, bifenthrin , bromacil, bromoxynil, butylate, cacodylic acid, captafol, captan, carbaryl, carbofuran, carbophenothion, carboxin, chloramben, chlordane, chlorobenzilate, chloropicrin, chlorothalonil, chlorpyrifos, chlropropham, Clethodim, clomazone, coumaphos, cyanazine, cyfluthrin, cypermethrin, dalapon, daminozide, DEET, DDT, deltamethrin, demeton-S-methyl, diazinon, dicamba, dichlorvos, diclofop-methyl, dicofol, dicrotophos, dienchlor, diflubenzuron, dimethoate, dimetomorph, dinocap, dinoseb, diphacinone, diquat Dibromide, disulfoton, diuron, dodine, ethylene dibromide, endosulfan, endothall, EPTC, esfenvalerate, ethephon, ethion, fenamiphos, fenitrothion, fenoxycarb, fenthion, fluazifop-p-butyl, flucythrinate, fluometuron, fluvalinate, folpet, fonofos, formothion, haloxyfop, heptachlor, hexachlorobenzene, hexazinone, hydramethylnon, imazalil, imazaquin, imazethapyr, imidacloprid, iprodione, isofenphos, lactofen, lambda-cyhalothrin, lindane, linuron, malathion, mancozeb, maneb, mecoprop, metalaxyl, metaldehyde, methamidophos, methidathion, methomyl, methoprene, methoxychlor, methyl bromide, methyl parathion, metiram, metolachlor, metribuzin, metsulfuron-methyl, mevinphos, molinate, monocrotophos, naled, napropamide, nicosulfuron, oryzalin, oxamyl, oxyfluorfen, paraquat, parathion, pendimethalin, pentachlorophenol, permethrin, phorate, phosalone, phosmet, picloram, primisulfuron-methyl, prometryn, pronamide, propanil, propazine, propetamphos, propoxur, pyrethrins and pyrethroids, quintozene, quizalofop-p-ethyl, resmethrin, rotenone, ryania, scilliroside, sethoxydim, simazine, streptomycin, sulfometuron-methyl, tebuthiuron, temephos, terbacil, terbufos, terbutryn, thiabendazole, thiram, triadimefon, triallate, trichlorfon, triclopyr, trifluralin, triforine, validamycin, vernolate, vinclozolin, warfarin, zineb, and ziram.

Liquid pheromones or solid pheromones dissolved in a carrier liquid can also be intermixed with the polymeric matrixes described herein to produce, for example, articles that can serve as baits or lures in insect traps, fishing lures, rodent traps, and the like. Pheromones are typically six-to-twenty carbon atom esters, aldehydes, alcohols and ketones and for that reason resemble fragrance compounds and can be immobilized as described earlier for fragrance compounds. There are many hundreds of such compounds identified for many animal and insect species, many of which are not considered pests. Representative examples that can be used in the articles described herein include, for example, E or Z-13-octadecenyl acetate; E or Z-11-hexadecenal; E or Z-9-hexadecenal; hexadecanal; E or Z-11 hexadecenyl acetate; E or Z-9-hexadecenyl acetate; E or Z-11-tetradecenal; E or Z-9-tetradecenal; tetradecanal; E or Z-11-tetradecenyl acetate; E or Z-9-tetradecenyl acetate; E or Z-7-tetradecenyl acetate; E or Z-5-tetradecenyl acetate; E or Z-4-tridecenyl acetate; E or Z-9-dodecenyl acetate; E or Z-8 dodecenyl acetate; E or Z-5-dodecenyl acetate; dodecenyl acetate; 11-dodecenyl acetate; dodecyl acetate; E or Z-7-decenyl acetate; E or Z-5-decenyl acetate; E or Z-3-decenyl acetate; octadecanal, Z or E, Z or E 3,13-octadecadienyl acetate; Z or E, Z or E 2,13-octadecdienyl acetate; Z, Z or E-7,11-hexadecadienyl acetate; Z, E 9,12-tetradecadienyl acetate; E, E-8,10-dodecadienyl acetate; Z, E 6,8-heneicosadien-11-one; E, E 7,9-heneicosadien-11-one; Z-6-henicosen-11-one; 7,8-epoxy-2-methyloctadecane; 2-methyl-7-octadecene, 7,8-epoxyoctadecane, Z,Z,Z-1,3,6, 9-nonadecatetraene; 5,11-dimethylheptadecane; 2,5 -dimethylheptadecane; 6-ethyl-2,3-dihydro-2-methyl-4H-pyran-4-one; methyl jasmonate; alpha-pinene; beta-pinene; terpinolene; limonene; 3-carene; p-cymene; ethyl crotonate; myrcene; camphene; camphor; 1,8-cineole; alpha-cubebene; allyl anisole; undecanal; nonanal; heptanal; E-2-hexenal; E-3-hexenal; hexanal; verbenene; verbenone; verbenol; 3-methyl-2-cyclohexenone; 3-methyl-3-cyclohexenone; frontalin; exo and endo brevicomin; lineatin; multistriatin; chalcogran; 7-methyl-1,6-dioxaspiro(4.5-decane,4,8-dimethyl-4(E),8(E)-decadienolide; 11-methyl-3(Z)-undecenolide; Z-3-dodecen-11-olide; Z,Z-3,6-dodecen-11-olide; Z-5-tetradecen-13-olide; Z,Z-5,8-tetradecen-13-olide; Z-14-methyl-8-hexadecenal; 4,8-dimethyldecanal; gamma-caprolactone; hexyl acetate; E-2-hexenyl acetate; butyl-2-methylbutanoate; propylhexanoate; hexylpropanoate; butylhexanoate; hexylbutanoate; butyl butyrate; E-crotylbutyrate; Z-9-tricosene; methyl eugenol; alpha-ionone; 4-(p-hydroxyphenyl)-2-butanone acetate; E-beta-farnasene; nepetalactone; 3-methyl-6-isopropenyl-9-decenyl acetate; Z-3-methyl-6-isopropenyl-3,9-decadienyl acetate; E or Z-3,7-dimethyl-2,7-octadecadienyl propionate; 2,6-dimethyl-1,5-heptadien-3-ol acetate; Z-2,2-dimethyl-3-isopropenyl cyclobutanemethanol acetate; E-6-isopropyl-3,9-dimethyl-5,8-decadienyl acetate; Z-5-(1-decenyl)dihydro-2(3H)-furanone; 2-phenethylpropionate; 3-methylene-7-methyl-7-octenyl propionate; 3,11-dimethyl-2-nonacosanone; 8-methylene-5-(1-methylethyl) spiro(11-oxabicyclo) 8.1.0-undecene-2,2-oxiran-3-one; 2-propylthietane; 3-propyl-1,2-dithiolane; 3,3-dimethyl-1,2-dithiolane; 2,2-dimethylthietane; E or Z-2,4,5-trimethylthiazoline; 2-sec-butyl-2-thiazoline; and isopentenyl methyl sulfide. Specific pheromones include the following: 8-methyl-2-decyl-propionate; 14-methyl-1-octadecene; 9-tricosense; tridecenyl acetate; dodecyl acetate; dodecenyl acetate; tetradecenyl acetate; tetradecadienyl acetate; hexadecenyl acetate; hexadecadienyl acetate; hexadecatrienyl acetate; octadecenyl acetate; dodecadienyl acetate; octadecadienyl acetate; and Z,E-9,12-tetradecadiene-1-ol.

The active liquid can be a liquid form of the active ingredient, or can be a solid, liquid or gaseous form of the active ingredient that is dissolved (contained) and diluted by a carrier liquid (diluent). In some examples, the active liquid can include or consist of water and an active agent dissolved in the water. Alternatively, the active liquid can include or consist of an organic liquid and an active agent dissolved in the liquid.

Examples of active ingredients contained in the active liquid can be therapeutically active ingredients (for humans or animals) such as medicines, drugs, pharmaceuticals, bioceuticals which are optionally combined with a biologically-acceptable carrier. Further, examples of the active ingredient contained in the active liquid can be biological compound such as amino acids, vitamins, carbohydrates, and/or steroids. Examples of biological compounds include biopolymers, biocopolymers, or chimera comprising DNA, RNA, oligonucleotides, modified DNA, modified RNA, proteins, polypeptides, and modified polypeptides.

Additional components for use in the polymeric matrixes include, for example, plasticizers, diluents, accelerators, retardants, tackifiers, fillers, and colorants. Phthalates, benzoates, salicylates, and lactate esters, alcohols, polyols, poly(alkylene glycol)s and alkyl and aryl ethers of alcohols, polyols and poly(alkylene glycols) are examples of useful plasticizers/diluents. These increase product flexibility, improve active release, and lower product cost. Reactive diluents and inert diluents can also be used to lower the initial blend viscosity. Possible diluents include, but are not limited to, various mono- and diglycidyl ethers, glycols, and N-methyl pyrolidinone. Phenols, such as nonyl phenol and 2,4,6-tris(dimethylaminomethyl)phenol, are examples of known accelerators of the epoxy-amine curing reaction that can shorten the time needed to cure the air fresheners described herein. Reaction accelerators include, for example, any alcohol-containing compound and/or water and/or mixtures thereof. Further, resins such as rosin esters and polyterpenes can be dissolved in the epoxy or the diluent/plasticizer to add tack to the final product. Examples of suitable resins include SYLVATAC, SYLVARES, and SYLVALITE, commercially available from Arizona Chemical (Jacksonville, Fla.).

The compositions and/or articles described herein can be made by reacting (for example, by contacting, mixing, or blending) a compound having at least two functional groups selected from epoxy, isocyanate, anhydride, and acrylate with a polyamine in the presence of an active liquid. The resultant mixture, prior to and after curing, can be homogeneous. Such contacting, mixing, and blending of the reactive components and active liquid (i.e., the reacting step) can occur at a temperature from 10-50° C. In some examples, the reacting step occurs at room temperature. In other examples, the reacting step can occur, for example, at 10° C., 15° C., 20° C., 25° C., 30° C., 35° C., 40° C., 45° C., or 50° C., inclusive of all ranges and subranges there between The components and optional ingredients can be added in any order. In some examples, the active liquid is added before the matrix-forming reaction proceeds to a point where its high viscosity and increasing elasticity precludes a blending operation. When the amine is a solid, it can first be dissolved in diluent liquid, in the active liquid, or in a mixture of both.

Temperature and blending conditions can be controlled so as to preclude premature curing, that is extensive curing during the contacting, mixing, or blending step. The mixture can become a homogeneous thermoset solid thereafter. Curing temperatures can differ from blending operation temperatures and can be in the range of from 10-100° C., for example, 10° C., 20° C., 30° C., 40° C., 50° C., 60° C., 70° C., 80° C., 90° C., and 100° C., inclusive of all ranges and subranges there between.

Curing rate is a function of at least six factors: curing temperature, functional group and amine group concentrations, ratio of these, structure of the amine, accelerator/retardant concentration, and composition of the fragrance oil/diluent. Accordingly, cure times can vary widely.

Mixing and/or curing can occur within a mold. For example, a low temperature procedure can include blending at room temperature, pouring the blend into a mold, sealing it, and allowing the blend to stand at room temperature. Such a procedure can take from a few minutes to a few days depending on the functional groups chosen and the reaction conditions. For example, the isocyanate-amine matrix reacts significantly faster than the epoxy-amine matrix. Another example is a pre-curing procedure useful more for the epoxy-amine matrix, which can include blending at room temperature, sealing tightly, heating to 70° C. for 30-90 minutes to obtain a partial cure but not gelling the composition, then pouring the resultant partial cure into a mold, letting it cool and stand at room temperature. Such a procedure can take from an hour to two days. Finally, another example is a high temperature procedure which can include blending at room temperature, pouring into a pouch or mold, sealing it tightly, and heating it to a temperature ranging from 60 to 100° C. Such a procedure can take from a few minutes to a few hours.

The steps of the methods described herein can be performed in any order and additional steps can be added. In addition, the curing time can range from 0.01 hour to 60 hours (e.g., from 5 minutes to 20 hours or from 10 minutes to 100 minutes). In some examples, the curing time can be 10 hours, 20 hours, 30 hours, 40 hours, 50 hours, 60 hours, 70 hours, 80 hours, 90 hours, or 100 hours, inclusive of all ranges and subranges there between.

In some examples, the method includes blending an active liquid, a liquid polyepoxy, and a liquid polyamine to form a mixture. Blending the components can occur at 10-40° C. However, the blending can be performed so as not to cause a loss of any temperature-sensitive active component. The temperature of blending can be 10° C., 15° C., 20° C., 25° C., 30° C., 35° C., or 40° C., inclusive of all ranges and subranges there between. When an epoxy-containing compound is used, the temperature of curing can be room temperature, i.e. 25° C., but can be higher, depending on the temperature sensitivity of the active liquid component and its volatility. If the active liquid does not degrade readily and the curing is performed in a sealed mold, the curing temperature, for example, can be about 60° C. At this temperature, curing for a typical formulation takes place in about 3-6 hours, or less if an accelerator is used.

In some examples, the methods described herein include blending an active liquid, a liquid diluent, a liquid polyisocyanate, and a liquid polyamine to form a mixture that cures to a liquid-immobilized polyurea composition. Blending the components can occur, for example, at 10-40° C. However, the blending can be performed so as not to cause a loss of any temperature-sensitive active component. The temperature of blending can be 10° C., 15° C., 20° C., 25° C., 30° C., 35° C., or 40° C., inclusive of all ranges and subranges there between.

In some embodiments, a catalyst is not present in the reaction between a polyamine and an isocyanate. However, even in the absence of a catalyst, the reaction between the polyamine and the isocyanate can be rapid at room temperature. In these examples, a rate modifier (i.e., a retardant) can be used to slow the reaction, allowing ample time for the ingredients to be blended and poured into a mold. Useful rate modifiers include, for example, aldehydes such as those normally present in common essential oils and fragrance oils. Other rate modifiers include those that are either bland in odor or those that can enhance the odor of the active liquid. Examples of useful retardants include aromatic aldehydes such as benzaldehyde, vanillin, and salicylaldehyde; α,β unsaturated aromatic aldehydes such as cinnamic aldehyde and methyl cinnamic aldehyde; terpenic aldehydes such as citral, cyclocitral, and citronellal; and C₄-C₁₈ aliphatic and cycloaliphatic aldehydes such as isobutyraldehyde, lyral, 2-phenyl propionaldehyde, and the like. While a retardant described above can be used when an isocyanate-containing compound is used, such a retardant can be optionally utilized in any of the methods described herein.

An aldehyde rate modifier retards the rate of the reaction by reacting with the polyamine to form a “blocked” amine in the form of an imine. In some embodiments, this reaction can be performed at room temperature. The rate of this reaction depends on several factors, such as the concentration of the aldehyde; whether the aldehyde is aliphatic or aromatic, or linear or branched; the functionality of the side chain(s); the acidity/alkalinity of the side chain(s); the electron donating or accepting capacity of the side chain(s); steric factors; and other factors. In these reactions, the aldehyde and polyamine reactants are in equilibrium with the imine and water is the by-product. When less than a stoichiometric amount of aldehyde is present, the reaction that generates the imine can proceed until all of the available aldehyde has interacted with the amine. In these reactions, unreacted amine can be present due to the reversible reaction and the value of the equilibrium constant. Upon addition of the isocyanate, the amine can react with the isocyanate, thus driving the equilibrium towards generating more amine. As the matrix-forming reaction proceeds, there can be less amine in the system relative to aldehyde, which favors blockage. Thus, an effective level of amine can be less than a stoichiometric amount. The presence of water forces the reversal of the process, so water can be used as an accelerator, negating the effect of aldehyde. Shown below is a table displaying the influence of the type and use level of aldehydes on the set time for isocyanate-polyamine reactions.

| | Aldehyde. | | | | | | Total | Time | Aldehyde/ |
|---|---|---|---|---|---|---|---|---|---|
| | Wt % | Aldehyde Eq. | Poly | Aldehyde. | MIBK Solvent | Polyisocyanate | Weight | To Set | Amine |
| ALDEHYDE | On Total | Wt. | Amine (g) | (g) | (g) | (g) | (g) | (min.) | (Eq. Ratio) |
| Blank (no aldehyde) | 0 | 156.22 | 4.7 | 0 | 4.7 | 0.49 | 9.9 | Instant | 0 |
| para-chlorobenzaldehyde | 1.4 | 140.57 | 4.7 | 0.14 | 4.7 | 0.49 | 10 | 17 | 0.37 |
| 2,4-dichlorobenzaldehyde | 1.74 | 175.01 | 4.7 | 0.175 | 4.7 | 0.49 | 10.1 | 23 | 0.37 |
| Citral + p-anisaldehyde | 3.83 | 152.24 | 4.7 | 0.394 | 4.7 | 0.49 | 10.3 | >400 | 0.30 + 0.65 |
| p-anisaldehyde | 2.68 | 136.15 | 4.7 | 0.272 | 4.7 | 0.49 | 10.2 | 50 | 0.75 |
| p-anisaldehyde | 3.02 | 136.15 | 4.7 | 0.308 | 4.7 | 0.49 | 10.2 | 400 | 0.85 |
| p-anisaldehyde | 5.24 | 136.15 | 4.7 | 0.547 | 4.7 | 0.49 | 10.4 | ca. 800 | 1.5 |
| 2,4-dichlorobenzaldehyde | 1.74 | 175.01 | 4.7 | 0.175 | 4.7 | 0.49 | 10.1 | 23 | 0.37 |
| 2,4-dichlorobenzaldehyde | 1.84 | 175.01 | 4.7 | 0.185 | 4.7 | 0.49 | 10.1 | 120 | 0.4 |
| 2,4-dichlorobenzaldehyde | 3.42 | 175.01 | 4.7 | 0.35 | 4.7 | 0.49 | 10.2 | 1110 | 0.75 |
| Alpha- | 5.54 | 216.33 | 4.7 | 0.58 | 4.7 | 0.49 | 10.5 | Instant | 1 |
| hexylcinnamaldehyde | | | | | | | | | |
| Alpha- | 6.83 | 216.33 | 4.7 | 0.725 | 4.7 | 0.49 | 10.6 | 1 | 1.25 |
| hexylcinnamaldehyde | | | | | | | | | |
| Alpha- | 8.09 | 216.33 | 4.7 | 0.87 | 4.7 | 0.49 | 10.8 | 10 | 1.5 |
| hexylcinnamaldehyde | | | | | | | | | |
| Alpha- | 9.27 | 216.33 | 4.7 | 1.01 | 4.7 | 0.49 | 10.9 | 19 | 1.75 |
| hexylcinnamaldehyde | | | | | | | | | |
| Alpha- | 10.46 | 216.33 | 4.7 | 1.155 | 4.7 | 0.49 | 11 | 28 | 2 |
| hexylcinnamaldehyde | | | | | | | | | |
| Citral | 1 | 152.24 | 4.7 | 0.1 | 4.7 | 0.49 | 10 | Instant | 0.25 |
| Citral | 1.22 | 152.24 | 4.7 | 0.122 | 4.7 | 0.49 | 10 | 100 | 0.3 |
| Citral | 1.49 | 152.24 | 4.7 | 0.15 | 4.7 | 0.49 | 10 | 210 | 0.37 |
| Citral | 1.98 | 152.24 | 4.7 | 0.2 | 4.7 | 0.49 | 10.1 | 588 | 0.49 |
| Citral | 2.98 | 152.24 | 4.7 | 0.304 | 4.7 | 0.49 | 10.2 | 1230 | 0.75 |
| Benzaldehyde | 1.06 | 106.12 | 4.7 | 0.106 | 4.7 | 0.49 | 10 | 27 | 0.37 |
| Benzaldehyde | 1.15 | 106.12 | 4.7 | 0.115 | 4.7 | 0.49 | 10 | 70 | 0.41 |
| Benzaldehyde | 1.3 | 106.12 | 4.7 | 0.13 | 4.7 | 0.49 | 10 | 360 | 0.46 |
| Benzaldehyde | 1.62 | 106.12 | 4.7 | 0.163 | 4.7 | 0.49 | 10.1 | 695 | 0.57 |
| Benzaldehyde | 2.1 | 106.12 | 4.7 | 0.212 | 4.7 | 0.49 | 10.1 | >700.0 | 0.75 |

The reaction between a polyamine and an isocyanate can be rapid at room temperature even in the absence of a catalyst. In some examples, a catalyst is not present. In these examples, a rate modifier (i.e., retardant) can be used to slow the reaction, allowing ample time for the ingredients to be blended and poured into a mold. Useful rate modifiers include, for example, aldehydes such as those normally present in common essential oils and fragrance oils. Others include those that are either bland in odor or enhance the odor of the active liquid. Examples of useful retardants are aromatic aldehydes such as benzaldehyde, vanillin, and salicylaldehyde; α,β unsaturated aromatic aldehydes such as cinnamic aldehyde and methyl cinnamic aldehyde; terpenic aldehydes such as citral, cyclocitral, and citronellal; and C₄-C₁₈ aliphatic and cycloaliphatic aldehydes such as isobutyraldehyde, lyral, 2-phenyl propionaldehyde and the like. While a retardant described above can be used when an isocyanate-containing compound is used, such a retardant can be optionally utilized in any of the methods described herein.

Another method for increasing cure times includes employing PAPA terminated with a carbonyl-substituted aromatic amine prepared according to the methods described herein. Shown below are the set times (i.e., the time from mixing to lack of flow) for four commercial fragrances immobilized at 50% concentration with matrix derived from the reaction of PAPA and DESMODUR N3300, the PAPA being terminated either by a non-aromatic primary amine or by a carbonyl-substituted aromatic amine, i.e., the PAPA terminated by reaction with para-aminobenzoic acid.

| | TIME TO SET (minutes) | |
|---|---|---|
| | PAPA with Non-Aromatic | PAPA with para-Amino- |
| | Primary Amine | Benzoic Acid |
| Fragrance Oil | Termination | Termination |
| Outdoor Breeze | ca. 0.2 | 400 |
| Tropical Splash | 34 | 420 |
| Clean Citrus | 39 | ca. 20 hours |
| Cotton Fresh | 54 | ca. 30 hours |

When an isocyanate-containing compound is utilized, the curing temperature can be room temperature, i.e. 25° C., but can be higher or lower, depending on the cure time desired. For example, if the active liquid does not degrade readily and a very rapid cure is desired, the curing can be carried out in a sealed mold, and at a curing temperature of about 50° C. At room temperature, curing for a typical formulation based on PAPA terminated by a primary aliphatic amine and carried out in the presence of little or no retardant, typical setting times are from less than 1 second to about 30 minutes. The time can be 0.1 minute, 0.5 minute, 1 minute, 5 minutes, 10 minutes, 20 minutes, or 30 minutes, including any and all ranges and subranges there between. Curing at room temperature for a typical formulation based on the carbonyl-substituted aromatic amine terminated polyamine can take place in from about 10 minutes to over 2 days when carried out in the presence of retardant but can be in the range 20-600 minutes in the absence of retardant. The time can be 20 minutes, 50 minutes, 100 minutes, 200 minutes, 300 minutes, or 600 minutes, including any and all ranges and subranges there between.

Further described herein are articles including the compositions described herein. In some examples, the articles further include a support material that can optionally be a porous support material. The articles described herein can include the gelled compositions. Examples of such articles include, but are not limited to, medicinal devices having an active liquid that is medicinally active, pesticide devices having an active liquid that is a pesticide, laundry care devices having an active liquid for laundry care (i.e., softener, fragrance, conditioner, cleaner, anti-stain, surface treating, and the like), or air freshener having an active liquid that is a fragrance. In some examples, the articles described herein can include the compositions in the form of aqueous dispersions. Examples of these articles include sun care products, skin care products, air fresheners, laundry fragrance sheets, laundry fabric softener sheets, laundry anti-static sheets, storage fragrance articles, pharmaceutical distribution articles, nutraceutical distribution articles, bioceutical distribution articles, moldicide distribution articles, bactericide distribution articles, pesticide distributions, decorative articles, biomedical sensors, and/or analytical devices.

The articles described herein can be processed into any desired shape that is appealing to a potential consumer. Such shapes can be 3-D shapes formed in a mold or a flat shape stamp-cut from pre-formed thin sheets. Shapes can include those geometrical in nature, e.g., triangular, square, circular, spherical, oval, regular geometric figure, irregular geometric figure, etc. For example, air care articles can have an immense variety of geometric and artistic shapes such as, but not limited to, disks, rings, cylinders, squares, rectangles, pentagons, hexagons, stars, hearts, hemispheres, spheres, cubes, flowers, animals, letters, numbers, logos, trademarks, and faces. Such shapes are limited only by methods known to make appropriate-shaped molds.

These articles can be colored with soluble dyes or with pigments. These colorants can be dissolved or dispersed prior to final mixing of the reactive components. These colorants can be conventional, fluorescent, pearlescent, temperature-sensitive, light-sensitive, pH-sensitive, or moisture sensitive. The latter four colorants allow for the preparation of novelty products that change color as environmental conditions change or that signal the depletion of the active component in the article.

Because the composition prior to curing is fluid, it can be poured easily into such molds and thus take on exacting shapes such as dimples, curves, logos, etchings, and any other embossed or engraved image. This is especially advantageous if the article is designed to fit directly into a holder, to adhere to a surface of complex shape, for example, a body part, a curved surface such as a heated potpourri dish, light bulb, or the inside of a package.

Prior to curing insoluble matter can be suspended in the reactive mixture so that when cross-linked, the system traps the suspended matter. Suspended matter can be decorative items such as icons, beads, glitter, gems, shards and the like; botanicals such as leaves, seeds, stems, needles, nuts, and the like; insoluble powdered materials such as wax, sugar, coffee grounds, bait particles, insoluble plain, colored or flavored salts, water, glycerin, silicone fluids, and aqueous solutions of dyes, active materials, acids, bases and the like with or without the aid of a surfactant to stabilize the dispersion thus formed; or with air or other gas by a whipping action or other deliberate mixing with the gas to form bubbles in the matrix-forming fluid. Alternatively, gas can be generated inside the matrix-forming composition by chemical means, such as, for example, thermal decomposition of a nitrogen-, oxygen-, or carbon dioxide-generating substance. Examples of such compounds are carboxylic acids, azobis(isobutyronitrile), hydrogen peroxide, and sodium carbonate or bicarbonate. A carboxylic acid that can be used in this way is polymerized fatty acid.

In some examples, the article described herein can include the fragrance oil or other active liquid and components selected from those listed above as immobilized by the cross-linked matrix. In other examples, the article can consist of the immobilized liquid and a support, be it a container, bracket, or holder into which the mixture of the reactive components, actives and other liquids and optional components are poured before curing takes place or fitted after curing takes place.

If not poured into a container, the article after curing can be coated, printed, or otherwise decorated, wrapped or supported by a stand, plate, bowl, dish, bracket, holder, or other supporting device. If poured into a container, the container can be made of glass, ceramic, metal, paper, plastic, or any other oil-impermeable material and be in any convenient shape such as a cylinder, tube, bowl, dish, etc. The container can itself be shaped to fit into a holder, chamber, or receptacle designed to fit into a fragrance dispensing device that can be fitted with a heater, fan, blower, or other mechanical aid. If the article is intended to be heated, the heater can be external to the cross-linked matrix-immobilized active liquid or it can be internal, that is, surrounded by or embedded in the cross-linked article. An example of such a device is a reactive composition poured into a container threaded with resistive heating wires that, after the matrix cures, can be electrified, thus heating the cross-linked composition from within.

Similarly, the composition while still fluid can be impregnated into a porous material such as paper, cardboard, cellulose pad, cellulose pulp, felt, fabric, a porous synthetic foam, a porous ceramic, activated carbon, soil, diatomaceous earth, kieselguhr, sand, charcoal, silica, clay, and the like or coated onto a non-porous substrate included but not limited to plastic films, metallic foils, rubber, ceramics, wood, glass, and leather.

Similarly, the mixture of reactive components, active liquid and optional liquids, while still uncured, can be dispersed in water or other aqueous medium and the resulting emulsion optionally stabilized by means of a surfactant. Droplets of the composition described herein, thus emulsified, can then cure, resulting in a dispersion of solid gel particles. This can be considered a process for preparing encapsulated active oils in dispersed form. Such a material is a milky liquid and can, as such, be impregnated into a porous medium such as paper, cardboard, cellulose pad, cellulose pulp, felt, fabric, a porous synthetic foam, a porous ceramic, activated carbon, soil, diatomaceous earth, kieselguhr, sand, charcoal, silica, clay, and the like or coated onto a non-porous substrate included but not limited to plastic films, metallic foils, rubber, ceramics, wood, glass, and leather.

In some examples, a container can be nearly filled with a volatile active liquid and can then be filled up with and sealed by the composition described herein, thus trapping the volatile material behind a barrier or membrane of cross-linked matrix. Such an arrangement allows the reservoir of volatile liquid to be released very slowly and continuously as it diffuses through the barrier of liquid-impregnated matrix.

In some examples, the article components can be insoluble in water without losing any of the desired final properties (e.g., fragrance release, stability) so that the water can optionally serve some useful purpose if incorporated in the cross-linked composition such as causing shrinkage to indicate end-of-use-life or introduction of a water-soluble active ingredient such as a dye or a salt.

In some examples, the articles can be prepared by (1) blending the polyamine, the active liquid and any desired optional components including diluents, plasticizers, fillers, stabilizers, and colorants; (2) blending this mixture with the polyepoxy or polyisocyanate component optionally diluted with further amounts of plasticizers, fillers, stabilizers, and colorants; (3) pouring out the final blend as a sheet or slab or into a support, form, container, or mold; (4) optionally covering or sealing the poured blend to protect it from contaminants and prevent volatile components from evaporating; (5) optionally storing it until the blend cures; and (6) optionally removing the cured immobilized liquid article from the sheet, slab, form, container, or mold and cutting it to another shape or using it as made in the container.

When the article is an air freshener, it can be “active” and/or “passive”. Active air fresheners encompass relatively complex devices having moving parts such as heaters and fans to dispense concentrated or diluted aroma compounds or spray cans charged with aroma chemical, carrier liquid, and propellant. Active air fresheners require the occupant to dispense the material into the area to be treated. Passive air fresheners are available in many forms, but are in essence “fixed” liquid chemicals: a multi-component article including fragrance oil immobilized in and/or a solid support. The support material can be simple, e.g., a piece of cardboard, blotter paper, cotton, or other fibrous materials. The support material can be complex, e.g., an aqueous dispersion (gelatin) or a non-aqueous gel (gelled, e.g., by polyamide resin). The air fresheners can be transparent, but, in some embodiments, can be opaque.

In some examples, the article is a visually attractive solid air freshener, in particular a room, closet, drawer, bag, area, container, or car interior freshener, that is both transparent or nearly transparent (e.g. “frosted”) and robust. In these examples, the active liquid is an aromatic composition (i.e. fragrance oil, scent, or perfume). As used herein, the term “robust” means that the article can be packaged inexpensively and handled without being deformed. The composition containing the aromatic material can be supported (i.e., in a container or holder) or free-standing. In particular, no special care is needed when the air freshener is taken out of its package or wrapper. Furthermore, the air freshener can resist changes in temperature, humidity, and exposure to light over the lifetime of its use or, with reasonable protection in a suitable package, over the lifetime of its storage and handling. The air care composition can also be free of syneresis (also known as “sweating”). The matrix material of the product is to be effectively non-toxic and not cause skin irritation if handled out of its storage wrapper. The air care composition lends itself readily to, but does not require the use of, porous powders, fabrics or fibers as a support for the fragrance oil.

The examples below are intended to further illustrate certain aspects of the methods and compounds described herein, and are not intended to limit the scope of the claims.

### EXAMPLES

### Example 1

Air freshener components (names and amounts listed below) including a small amount of green dye, which were weighed into a glass vial and stirred together at ambient temperature by hand with a wooden stir stick. A portion of the mixture (8.0 g) was then poured into a flat, rectangular, 2.50 inch×3.25 inch uncoated polystyrene mold:

Epoxy Resin: EPALLOY® 5001, 10.00 g; 55.1%

Hardener: 1,3-BAC, 3.55 g; 19.6%

Fragrance Oil: Belle Aire “Evergreen”, 4.55 g; 25.1%

Dye: Green, 0.05 g; 0.3%.

The next day the sample was firm, clear, tack-free, and flexible. It could be removed from the mold by hand with only a slight amount of sticking to the mold. Placed in a polyethylene “baggie” for storage at room temperature, it exhibited no syneresis, even after a number of weeks.

### Example 2

These air freshener components totaling 100 parts by weight were treated following the procedure of Example 1: EPALLOY® 5001 (53.6 parts), 1,3-BAC (19.0 parts), Belle Aire “Evergreen” fragrance oil (25.1 parts), nonyl phenol (2.2 parts). The resulting article after curing at room temperature for one day was transparent, firm, flexible and tack-free.

### Example 3

These air freshener components totaling 100 parts by weight were treated following the procedure of Example 1: Cyclohexane dimethanol diglycidyl ether (22.8 parts), EPON® 828 (22.8 parts), Huntsman T-403 polyamine (24.2 parts), Continental Aromatics “Country Meadow” fragrance oil (30.0 parts), plastic glitter 0.1 parts) and a trace of green dye. The resulting article after curing at room temperature for three days was transparent, firm, flexible, tack-free and exhibited ability to cling lightly to a flat vertical glass surface from which it could be easily removed and re-applied without marring the surface.

### Example 4

A polyamide polyamine was prepared by charging adipic acid (20.0 g, 274 meq acid), JEFFAMINE® T-403 polyamine (20 g, 132 meq amine) and Huntsman XTJ-500 (80 g, 254 meq. amine) to a 250 mL glass flask equipped with a stirrer and heating this charge to 210-220° C. under a stream of dry nitrogen. After holding this mixture under these conditions for 5 hours, the reaction mixture was discharged to a container. The product was a clear, viscous, nearly water-white liquid having an acid number of 1.4, an amine number of 42.2, and a Brookfield viscosity at 150° C. of 340 cP. A portion of this product (11.63 g) was dissolved in water (27.5 g) and then blended with a polyethyleneglycol diglycidyl ether (EEW of 195; 3.40 g). To a portion of this mixture (20.0 g) in a small plastic jar with a screw cap was then added fragrance oil (“Sunshine Fruits”, Firmenich fragrance oil #190196) and a few drops of Tween 80 surfactant, forming a milky emulsion which, after being capped and allowed to stand, gelled to an immobile firm homogeneous white solid that emitted the fragrance gradually after being un-capped.

### Example 5

To a commercial resealable polyethylene “baggie” was added components totaling 100 parts by weight: cyclohexane dimethanol diglycidyl ether (13.9 parts), EPON® 826 (13.9 parts), Arizona proprietary liquid triethylenetetraamine-based amido-amine #X54-327-004 (amine number of 349, acid number of 0.8, 22.2 parts), Atlas “Crisp Breeze” fragrance oil (50.0 parts), and a trace of blue dye. The “baggie” was massaged to blend the components for a few minutes, the air bubbles pressed out and the fluid mixture then stored lying flat at room temperature for one week. At that time the material was cross-linked to the point of being immobile, transparent, and flexible.

### Example 6

To a glass beaker containing a magnetic stir bar was charged Huntsman Surfonic®L24-5, a liquid ethoxylated alcohol surfactant (12.0 g), Atlas Products “Crisp Breeze” fragrance oil (8.0 g), Huntsman T-403 polyamine (8.4 g), FD&C #3 blue-green dye (0.4 g) and HELOXY® 48 epoxy resin (14.0 g). This mixture was heated to 58° C. for about 3 hours with stirring to nearly cure it and then poured into a cylindrical mold and allowed to cool. After the material stood about three days at room temperature it was removed from the mold as a slightly rubbery, firm solid.

### Example 7

These air freshener components totaling 100 parts by weight were blended at room temperature: cyclohexane dimethanol diglycidyl ether (25.3 parts), EPON® 828 (17.2 parts), Arizona proprietary polyamido-amine hardener #X54-327-004 (34.5 parts), Continental Aromatics “Ocean” fragrance oil (23.0 parts), and a trace of green dye. This blend was held for about 45 minutes at about 67° C., at which time it was allowed to cool to room temperature. It was, at this stage, quite viscous, but could still be poured and stirred. To this partially cross-linked intermediate was added with gentle distribution through the mass approximately two dozen ¼ colored foil hearts. The resulting article after curing at room temperature for three days was firm, flexible, and tack-free with the foil hearts clearly visible suspended uniformly inside it.

### Example 8

These components totaling 100 parts by weight were treated following the procedure of Example 1: poly(propylene glycol) diglycidyl ether (13.0 parts), EPON® 828 (22.0 parts), Arizona UNI-REZ® 2801 amido-amine (14.0 parts), “Vanilla” fragrance oil from Aromatic Flavors and Fragrances, dipropyleneglycol benzoate (19.5 parts) and commercial ground coffee (29.5 parts). The resulting article after curing was firm, slightly flexible, non-tacky. The coffee grounds were uniformly distributed and gave the article a rich brown, opaque appearance, smooth at the bottom where the mold was smooth and rough on top where the grounds were allowed to settle freely.

In the following examples, abbreviations are as follows:
CHDA is 1, 4 cyclohexane dicarboxylic acid from Eastman Chemical;
Empol is EMPOL® 1008 dimer acid supplied by Cognis Corporation;
Unidyme is UNIDYME® 18 dimer acid supplied by Arizona Chemical Company;
T-403 is JAFFAMINE® T-403 poly(alkyleneoxy) diamine supplied by Huntsman Corporation;
D-400 is JEFFAMINE®D-400 poly(alkyleneoxy) diamine also from Huntsman;
D-2000 is JEFFAMINE®T-2000 poly(alkyleneoxy) diamine also from Huntsman;
V-551 is VERSAMINE® 551 dimer diamine supplied by Cognis Corporation;
N-3300 is DESMODUR® N-3300 or N-3300A, Bayer Corporation, Industrial Chemicals Division;
N-3800 is DESMODUR® N-3800, also from Bayer;
Z-4470 is DESMODUR® Z4470, also from Bayer.

### Example 9

A polyamide polyamine was prepared by charging EMPOL®1008 polymerized fatty acid (63.0 g, 219 meq acid), JEFFAMINE®T-403 polyamine (18 g, 118 meq amine) and JEFFAMINE®D-400 (45 g, 205 meq. amine) to a 250 mL glass flask equipped with a stirrer and heating this charge to 210-220° C. under a stream of dry nitrogen. After holding this mixture under these conditions for 5 hours, the reaction mixture was discharged to a container. The product was a clear, viscous, nearly water-white liquid having an acid number of 0.3, an amine number of 41.8, a weight average molecular weight of 2,270, and a Brookfield viscosity at 150° C. of 204 cP.

A solution was prepared by warming 10.0 g of this polyamide polyamine with 5.0 g FINSOLV® TN benzoate ester and 10.0 g fragrance oil (“Linen Fresh”, Wessel Fragrances), cooled to room temperature and blended thoroughly with a mixture of DESMODUR® Z4470 and 5.1 g additional fragrance oil. To the composition was then added a small amount of red dye and red glitter. A few minutes later about 25 g of this final formulation was poured into a flat, circular rose-shaped silicone rubber mold and the remainder retained in a jar. A total of 33 minutes after the component were blended, the retained material was set to an immobile gel. After standing at room temperature for 16 hours, the immobilized fragrance oil article was removed from the mold. It did not adhere to the mold, was non-tacky, had the exact flower shape of the mold, exhibited a uniform color and distribution of glitter, and could be handled without breaking up. It also exhibited excellent cling to a variety of vertical surfaces including glass and plastic film.

### Examples 10-15

Polyamide polyamines were prepared according to the procedure of Example 9 by charging acids and amines of the types listed in the TABLE A (below) in the weight percentages indicated to a reactor and heating the charge to 200-220° C. under a stream of dry nitrogen for about 4-5 hours and discharging the product. Products properties were then measured and are also recorded in TABLE 1.

TABLE 1

| | EXAMPLE NUMBER | | | | | |
|---|---|---|---|---|---|---|
| | Example 10 | Example 11 | Example 12 | Example 13 | Example 14 | Example 15 |
| COMPONENTS | | | | | | |
| DiAcid | Adipic Acid | Empol | Empol | Empol | CHDA | Unidyme |
| Diamine | T-5000 | T-403 | T-403 | T-403 | T-403 | D-2000 |
| Co-DiAmine | — | D-400 | D-400 | XTJ-500 | D-400 | Piperazine |
| Third Diamine | — | D-2000 | D-2000 | — | D-2000 | |
| COMPONENTS (Wt. %) | | | | | | |
| DiAcid | 2.0% | 41.2% | 30.8% | 43.3% | 18.7% | 82.3% |
| Diamine | 98.0% | 9.6% | 4.2% | 12.6% | 17.8% | 2.1% |
| Co-DiAmine | 0.0% | 24.6% | 16.7% | 44.1% | 35.5% | 15.6% |
| Third Diamine | 0.0% | 24.6% | 48.3% | 0.0% | 28.0% | 0.0% |
| PRODUCT PROPERTIES | | | | | | |
| Neutralization | 194.4% | 139.5% | 141.5% | 148.2% | 141.1% | 131.7% |
| Acid Number | 0.4 | 0.5 | 0.4 | 0.4 | 1.4 | 0.6 |
| Amine Number | 12.2 | 27.1 | 22.6 | 42.4 | 44.6 | 14.1 |
| Color | Pale yellow | Pale | Pale | Off-White | Pale | Amber |
| | | yellow | yellow | | yellow | |
| Softening Point (R&B, ° C.) | Liquid | Liquid | Liquid | Liquid | 128 | Liquid |
| Viscosity At 150° C. | 770 | 391 | 141 | 190 | 290 | 481 |
| Wt. Aver. Mol. Wt. | 6,150 | 2,150 | 17,780 | 5,650 | 1,720 | 33,760 |

Immobilized fragrance oils were prepared by warming a mixture of 2.0 grams PAPA of the example and 2.0 grams fragrance oil to about 55° C. and then blending the warm mixture by hand with a stir stick. Test fragrances were: “Ocean” (Continental Aromatics), “Linen Fresh” (Wessel Fragrances), and “Cherry” (Aromatic Flavors and Fragrances). After blending, one equivalent of isocyanate hardener dissolved in an equal weight of oil was added with manual stirring, a stopwatch was started, and the mixture monitored for its consistency. When the mixture no longer could flow under its own weight, the time (in minutes) was noted as the “gel time”. TABLE 2 shows that all of these polyamide polyamines were effective in immobilizing the target oils when cross-linked with polyisocyanates. Gel times were short but not so short as to preclude the preparation of useful articles and followed the consistent pattern: Ocean<Linen Fresh<Cherry.

TABLE 2

| GEL COMPONENTS | | POLYAMIDE POLYAMINE OF EXAMPLE | | | | | | |
|---|---|---|---|---|---|---|---|---|
| Fragrance Oil Type | Hardener | No. 9 | No. 10 | No. 11 | No. 12 | No. 13 | No. 14 | No. 15 |
| Ocean | N-3300 | 6.5 | 15 | 10 | 40 | 8.5 | 10 | 73 |
| Linen Fresh | N-3300 | 9 | 24 | 13 | 55 | 10 | 13 | 76 |
| Linen Fresh | Z-4470 | 33* | 44 | 22 | nd | nd | nd | nd |
| Cherry | N-3300 | 75 | 170 | 95 | 335 | 87 | nd | nd |

*40% polyurea- see Example 9 for conditions

### Examples 16-20

Polyamide polyamines (PAPA) were prepared according to the procedure of Example 9 by charging acids and amines of the types listed in the TABLE C in the weight percentages indicated to a reactor and heating the charge to 200-220° C. under a stream of dry nitrogen for about 5 hours and discharging the product. Products properties were then measured and are also recorded in TABLE 3.

TABLE 3

| EXAMPLE | No. 16 | No. 17 | No. 18 | No. 19 | No. 20 |
|---|---|---|---|---|---|
| COMPONENTS | | | | | |
| DiAcid | Empol | Adipic Acid | Adipic Acid | Empol 1008 | Unidyme |
| Triamine | T-403 | T-403 | T-403 | — | — |
| Diamine | D-400 | XTJ-500 | D-400 | D-400 | V-551 |
| Third Amine | D-2000 | — | D-2000 | D-2000 | — |
| WEIGHT % | | | | | |
| DiAcid | 30.6% | 18.2% | 15.2% | 36.7% | 41.7% |
| Triamine | 5.0% | 9.1% | 7.6% | — | — |
| Diamine | 16.5% | 72.7% | 38.6% | 22.9% | 58.3% |
| Third Amine | 47.9% | — | 38.6% | 40.4% | — |
| PROPERTIES | | | | | |
| Acid Number | 0.6 | 2.2 | 0.7 | 0.7 | 1.1 |
| Amine Number | 27.0 | 28.9 | 29.9 | 13.1 | 33.2 |
| Color | Colorless | Colorless | Colorless | Colorless | Amber |
| Viscosity | 106 | 393 | 198 | 1340 | 656 |
| [cP at 150° C.] | | | | | |
| Weight Aver. MW | 26380 | 12230 | 13490 | 31550 | 13180 |

Immobilized fragrance oils were prepared by warming a mixture of 2.0 grams polyamide polyamine of the example and 2.0 grams fragrance oil to about 55° C. and then blending the warm mixture by hand with a stir stick. Test fragrances were: Oceanside Mist, Tropical (Atlas Products), Spring Meadow, Country Wildflower, Ocean (Continental Aromatics), Linen Fresh (Wessel Fragrances), Yankee Home (Belle Aire), Mulberry and Cherry (Aromatic Flavors and Fragrances). After blending, one equivalent of isocyanate hardener dissolved in an equal weight of oil was added with manual stirring, a stopwatch was started, and the mixture monitored for its consistency. When the mixture no longer could flow under its own weight, the time (in minutes) was noted as the “gel time”. TABLE 4 shows that all of these polyamide polyamines were effective in immobilizing the target oils when cross-linked with polyisocyanates. Gel times were short but not so short as to preclude the preparation of useful articles and followed the consistent pattern:
Spring Meadow<Ocean<Tropical<Linen Fresh<Yankee Home<Mulberry<Cherry

TABLE 4

| | Polyamide Polyamine of Example | | | | |
|---|---|---|---|---|---|
| Fragrance Oil Type | No. 16 | No. 17 | No. 18 | No. 19 | No. 20 |
| Oceanside Mist | Nd | nd | nd | 41 | Nd |
| Spring Meadow | Nd | nd | nd | 42 | Nd |
| Country Wildflower | Nd | nd | nd | 75 | nd |
| Ocean | 32 | 14 | 18 | >180 | 4 |
| Tropical | 38 | nd | 29 | >180 | nd |
| Linen Fresh | 40 | 20 | 32 | 225 | 13 |
| Yankee Home | 80 | 27 | 51 | >180 | nd |
| Mulberry | 315 | 185 | 250 | nd | nd |
| Cherry | >420 | 360 | >300 | >180 | 240 |

### Example 21

A number of batches of a PAPA were prepared by the method of Example 9 using a charge (weight percentages in brackets) of either EMPOL® 1008 or UNIDYME® 12 (a low trimer content, hydrogenated dimer acid obtained from Arizona Chemical) [29.5%], T-403 [3.7%], D-400 [22.6%], and D-2000 [44.2%]. This polymer, used in Examples #22-35, typically had an amine number of 30-35 (equivalent wt. of 1,800-1,600), a weight-average molecular weight of 10,700-12,100, a number-average molecular weight of 4,300-4,900, and a viscosity at 150° C. of 40-70 cP.

### Example 22

This example illustrates the preparation of an air freshener in a simple geometric shape. To a glass mixing jar was charged 13.1 g of the Example 21 PAPA and 15 g of “Cotton Fresh” fragrance oil (Symrise Corp.) and the mixture was stirred gently for 15 minutes at ambient temperature. Blue dye (2 drops) was added to the mixture, turning the solution light blue. To this homogeneous mixture was then added 1.5 g of DESMODUR® N3300A. This mixture was then stirred until homogeneous, allowed to stand a few minutes to allow any air bubbles to dissipate, and 13 g total was poured into a rectangular-shaped flexible silicone mold of uniform length of 1.87 inches, height of 0.3 inches, and width of 1.0 inches. The set time was recorded at 28 minutes. The mixture was covered with polyethylene film and allowed to cure undisturbed for 24 hours. After this time the mold was stripped away from the cross-linked air freshener object that was now firm, flexible, transparent and non-tacky to the touch.

### Example 23

This example illustrates the preparation of an air freshener in a complex shape. To a glass mixing jar was charged 13.1 g of the Example 21 polyamine and 15 g of “Snuggle Type” fragrance oil (Alpha Aromatics) and the mixture was stirred gently for 15 minutes at ambient temperature. Red dye (3 drops) was added to the mixture, turning the solution light pink/red. To this homogeneous mixture was then added 1.5 g of DESMODUR® N3300A. This mixture was then stirred briefly (until homogeneous), allowed to stand a few minutes to allow any air bubbles to dissipate, l Og total was poured into a circular-shaped briochette flexible silicone mold of uniform top-width of 1.875 inches, height of 0.375 inches, and bottom-width of 1.625 inches. The set time was 6 minutes. The mixture was covered with polyethylene film and allowed to cure undisturbed for 24 hours. After this time the mold was stripped away from the cross-linked air freshener article that was now firm, flexible, transparent, and non-tacky to the touch.

### Example 24

This example illustrates the preparation of an air freshener in a complex shape. To a glass mixing jar was charged 19 g of the Example 21 polyamine and 20 g of “Tropical Splash” fragrance oil (obtained from Symrise Corp.) and the mixture was stirred gently for 15 minutes at ambient temperature. Blue dye (3 drops) was added to the mixture, turning the solution light green. To this homogeneous mixture was then added 2.0 g of DESMODUR® N3300A. This mixture was then stirred briefly (until homogeneous), allowed to stand a few minutes to allow any air bubbles to dissipate, 20 g total was poured into a scallop-shaped flexible silicone mold of uniform top-width of 2.375 inches, height of 0.125 inches, and bottom-width of 2.25 inches. The set time was recorded at 24 minutes. The mixture was covered and allowed to cure undisturbed for 24 hours. After this time the mold was stripped away from the cross-linked air freshener article that was now firm, flexible, transparent, and non-tacky to the touch.

### Example 25

This example illustrates the preparation of an air freshener containing suspended insoluble particles. To a glass mixing jar was charged 19 g of the Example 21 polyamine and 20 g of “Clean Citrus” fragrance oil (from Symrise Corp.) and the mixture was stirred gently for 15 minutes at ambient temperature. Yellow aluminum flake “glitter” (0.04 g) was added to the mixture. To this homogeneous mixture was then added 2.0 g of DESMODUR® N3300A. This mixture was then stirred briefly (until homogeneous), allowed to stand a few minutes to allow any air bubbles to dissipate, 18.0 g total was poured into a disk-shaped flexible silicone mold of uniform circumference of 9.75 inches, height of 0.75 inches, and width of 3.0 inches. The set time was recorded at 30 minutes. The mixture was covered and allowed to cure undisturbed for 24 hours. After this time the mold was stripped away from the cross-linked air freshener article that was now firm, flexible, transparent, and non-tacky to the touch and displayed a uniform distribution of glitter.

### Example 26

To a glass mixing jar was charged 19 g of the Example 21 polyamine and 20 g of “Sunshine Fruit” fragrance oil (Firmenich, Inc.) and the mixture was stirred gently for 15 minutes at ambient temperature. Green “glitter” (0.03 g) was added to the mixture. To this homogeneous mixture was then added 2.0 g of DESMODUR® N3300A. This mixture was then stirred briefly (until homogeneous), allowed to stand a few minutes to allow any air bubbles to dissipate, 28.0 g total was poured into a heart-shaped flexible silicone mold of uniform length of 2.5 inches, height of 0.3 inches, and width of 2.875 inches. The set time was recorded at 17 minutes. The mixture was covered and allowed to cure undisturbed for 24 hours. After this time the mold was striped away from the cross-linked air freshener object that was now firm, flexible, transparent and non-tacky to the touch and displayed a uniform distribution of glitter.

### Example 27

To a glass mixing jar was charged 19 g of the Example 21 PAPA and 20 g of “Mandarin Grapefruit” fragrance oil (obtained from Givaudan Corp.) and the mixture was stirred gently for 15 minutes at ambient temperature. Blue dye (1 drop) was added to the mixture, turning the solution light yellow/green. To this homogeneous mixture was then added 2.0 g of DESMODUR® N3300A. This mixture was then stirred briefly (until homogeneous), allowed to stand a few minutes to allow any air bubbles to dissipate, 31.0 g total was poured into a Bundt cake-shaped flexible silicone mold of uniform top-width of 1.75 inches, height of 0.75 inches, and bottom-width of 2.5 inches. The set time was recorded at 67 minutes. The mixture was covered and allowed to cure undisturbed for 24 hours. After this time the mold was stripped away from the cross-linked air freshener object that was now firm, flexible, transparent and non-tacky to the touch.

### Example 28

This example illustrates the preparation of an immobilized phase-transfer liquid. To a glass mixing jar was charged 10.4 g of the Example 21 polyamine and 18 g of 1-decanol (freezing point, 5-7° C.) as the active oil, 0.6 g benzaldehyde as odorant and cross-linking reaction retardant and the mixture was stirred gently for 15 minutes at ambient temperature. To this homogeneous mixture was then added 1.5 g of DESMODUR® N3300A. This mixture was then stirred briefly (until homogeneous), allowed to stand a few minutes to allow any air bubbles to dissipate and then 18.5 g total was poured into a truncated pyramid-shaped flexible silicone mold of uniform top-width of 0.75 inches, height of 0.75 inches, and bottom-width of 1.0 inches. The set time was recorded at 30 minutes. The mixture was covered and allowed to cure undisturbed for 24 hours. After this time the mold was stripped away from the cross-linked object that was now firm, flexible, transparent and non-tacky to the touch. When placed in a freezer. The object hardened but did not crack. When removed from the freezer and allowed to warm to room temperature, the object regained flexibility but remained a tough, firm clear, solid.

### Example 29

This example illustrates the preparation of a small air freshener for use in a purse or other small enclosed space): To a glass mixing jar was charged 5 g of the Example 21 polyamine and 5 g of “Ocean” fragrance oil (provided by Orlandi, Inc.) and the mixture was stirred gently for 15 minutes at ambient temperature. Blue dye (2 drops) was added to the mixture, turning the solution light blue. To this homogeneous mixture was then added 0.6 g of DESMODUR® N3300A. This mixture was then stirred briefly (until homogeneous), allowed to stand a few minutes to allow any air bubbles to dissipate, 5.0 g total was poured into a lozenge-shape polyethylene bulb mold of uniform middle-circumference of 1.5 inches, height of 1.625 inches, and top and bottom-width of 0.5 inches. The set time was 7 minutes. The mixture was sealed and allowed to cure undisturbed for 24 hours. After this time the mold was stripped away from the cross-linked air freshener object that was now firm, transparent, and non-tacky to the touch.

### Example 30

To a glass mixing jar was charged 28 g of the Example 21 polyamine and 30 g of “Country Garden” fragrance oil (Belle-Aire) and the mixture was stirred gently for 15 minutes at ambient temperature. Green dye (3 drops) and yellow sprinkles (0.02 g) were added to the mixture, turning the solution yellow/green. To this homogeneous mixture was then added 3.0 g of DESMODUR® N3300A. This mixture was then stirred briefly (until homogeneous), allowed to stand a few minutes to allow any air bubbles to dissipate, 50.0 g total was poured into a Half sphere-shaped flexible silicone mold of bottom-circumference of 7.25 inches, height of 1.0 inches, and bottom-width of 3.75 inches. The set time was 260 minutes. The mixture was covered and allowed to cure undisturbed for 24 hours. After this time the mold was stripped away from the cross-linked air freshener object that was now firm, flexible, transparent and non-tacky to the touch.

### Example 31

To a glass mixing jar was charged 30 g of the Example 21 polyamine and 30 g of “Cotton Fresh” fragrance oil (Symrise) and the mixture was stirred gently for 15 minutes at ambient temperature. Autumn leaves foil confetti (6 leaves) were added to the clear mixture. To this homogeneous mixture was then added 3.5 g of DESMODUR® N3300A. This mixture was then stirred briefly (until homogeneous), allowed to stand a few minutes to allow any air bubbles to dissipate, and 50 g total was poured into a glass jar of uniform circumference of 7.25 inches, height of 1.25 inches, and top and bottom-width of 2.25 inches. The set time was recorded at 28 minutes. The mixture was capped and allowed to cure undisturbed for 24 hours. After this time the mold was now firm, transparent, and smooth to the touch.

### Example 32

To a glass mixing jar was charged 37 g of the Example 21 polyamine and 40 g of “Lemon Citrus” fragrance oil (Alpha Aromatics) and the mixture was stirred gently for 15 minutes at ambient temperature. Green sprinkles (0.02 g) were added to the mixture, turning the solution yellow/green. To this homogeneous mixture was then added 4.0 g of DESMODUR® N3300A. This mixture was then stirred briefly (until homogeneous), allowed to stand a few minutes to allow any air bubbles to dissipate, 60.0 g total was poured into a lemon-shaped flexible silicone mold of uniform top and bottom-width of 0.75 inches, height of 2.75 inches, and middle-circumference of 5.5 inches. The set time was recorded at 42 minutes. The mixture was covered and allowed to cure undisturbed for 24 hours. After this time the mold was stripped away from the cross-linked air freshener object that was now firm, flexible, transparent and non-tacky to the touch.

### Example 33

To a glass mixing jar was charged 36 g of the Example 21 polyamine and 40 g of “Cherry Berry” fragrance oil (Belle-Aire) and the mixture was stirred gently for 15 minutes at ambient temperature. Red dye (3 drops) was added to the mixture, turning the solution red. To this homogeneous mixture was then added 4.0 g of DESMODUR® N3300A. This mixture was then stirred until homogeneous, allowed to stand a few minutes to allow any air bubbles to dissipate, 60.0 g total was poured into a rose flower-shaped flexible silicone mold of uniform top and bottom-width of 3.75 inches, height of 0.75 inches, and circumference of 12.25 inches. The set time was recorded at 155 minutes. The mixture was covered and allowed to cure undisturbed for 24 hours. After this time the mold was stripped away from the cross-linked air freshener object that was now firm, flexible, transparent, and non-tacky to the touch.

### Example 34

To a glass mixing jar was charged 19 g of the Example 21 polyamine and 20 g of “Cherry” fragrance (Atlas, Inc.) and the mixture was stirred gently for 15 minutes at ambient temperature. Red dye (3 drops) was added to the mixture, turning the solution red. To this homogeneous mixture was then added 2.0 g of DESMODUR® N3300A. This mixture was then stirred briefly (until homogeneous), allowed to stand a few minutes to allow any air bubbles to dissipate, 28.0 g total was poured into a hollow polyethylene golf ball mold of uniform circumference 5.25 inches. The set time was recorded at 75 minutes. The mixture was covered and allowed to cure undisturbed for 24 hours. After this time the mold was stripped away from the cross-linked air freshener object that was now firm, flexible, transparent, and non-tacky to the touch.

### Example 35

This example illustrates the preparation of a foamed article. To a glass mixing jar was charged 15 g of the Example 21 polyamine, 15 g of UNIDYME® 60 polymerized fatty acid (from Arizona Chemical) and 30 g of “Very Berry” fragrance oil (from Belmay Corp.) and the mixture was stirred gently for 15 minutes at ambient temperature, resulting in a slightly hazy solution. Red dye (3 drops) was added to the mixture, turning the solution red. To this homogeneous mixture was then added 4.0 g of DESMODUR® N3300A. This mixture was then stirred briefly (until homogeneous), allowed to stand a few minutes to allow any air bubbles to dissipate, 40 g total was poured into a baking cup paper mold of uniform top and bottom-width of 2.0 inches, height of 1.25 inches, and circumference of 7.5 inches. The set time was 8 minutes. The mixture was allowed to cure undisturbed for an additional 24 hours. During this time the object became filled with trapped bubbles (foam) and doubled in size, forming a rounded crown. This foam air freshener was now firm and non-tacky to the touch. When compressed (squeezed), it returned to its rounded shape.

### Example 36

A number of batches of a polyamide polyamine terminated with a carbonyl-substituted aromatic amine were prepared by charging (weight percentages in brackets) of PRIPOL® 1009 hydrogenated dimer acid [24.0], para-aminobenzoic acid [5.0], JEFFAMINE® D-2000 [54.0], JEFFAMINE® D-400 [11.5], and JEFFAMINE® T-403[5.5] to a 3L glass round-bottomed reactor equipped with an overhead mechanical stirrer and heating this charge to 215° C. under a stream of dry nitrogen. After holding this mixture under these conditions for about 25 hours, the reaction mixture was discharged to a container. The product was a clear, viscous, slightly yellow liquid. This polymer had a titrated amine number in the range 13-15 (non-potentiometric method, or 30-35 by potentiometric titration, amine reactive equivalent wt. of 1,800-1,600), a weight-average molecular weight of 13,000-14,000, a number-average molecular weight of 4,500-5,500, and a viscosity at 130° C. of 250 cP. This material was used in a series of tests of immobilizing, at the 30 weight % use level, liquid test media (70% by weight), free of active, catalyst, or retardant. The results (TABLE, 5 below) demonstrate that set times can vary up to about 1 day for such a modified PAPA even in the absence of retardant aldehyde. The data also demonstrate the accelerating effect of the use of an alcoholic diluent, such as a polypropylene glycol or its alkyl ether, on the cure rate.

TABLE 5

| | Set Time | Upon Curing | Syneresis |
|---|---|---|---|
| Test Liquid Medium | (Minutes) | Appearance | (after 4 days) |
| Dipropylene Glycol | 60 | Hazy | Slight |
| | | | syneresis |
| Isostearyl Alcohol | 60 | Hazy | No syneresis |
| Tripropylene Glycol | 66 | Slight | Significant |
| | | Haze | syneresis |
| Dipropylene Glycol | 90 | Clear | No syneresis |
| Mono Methyl Ether | | | |
| Castor Oil | 105 | Slight | No syneresis |
| | | Haze | |
| Methyl Salicylate | 400 | Clear | No syneresis |
| FINSOLV TN Benzoate | 440 | Clear | No syneresis |
| Ester | | | |
| Dibutyl Adipate | 1014 | Clear | No syneresis |
| Dipropylene Glycol | 1245 | Clear | No syneresis |
| Dimethyl Ether | | | |
| Diethyl-m-toluamide | 1470 | Clear | No syneresis |
| (DEET) | | | |
| Isophorone | 1845 | Clear, | No syneresis |
| | | yellow | |

### Example 37

This example illustrates the preparation of another type of polyamide polyamine terminated with a carbonyl-substituted aromatic amine. The procedure of Example 36 was followed using a charge (weight percentages in brackets) of T-5000 [92.9] and para-aminobenzoic acd [7.1]. This polymer, used in Examples #38-41, had an amine equivalent weight of 1.950.

### Example 38

This example illustrates the preparation of an article containing liquid fragrance trapped behind a membrane of matrix. To a glass mixing jar was charged 5.0 g of the Example 37 polyamine and 5.0 g of FINSOLV® TN and the mixture was stirred gently for 15 minutes at ambient temperature. To this homogeneous mixture was then added 0.6 g of DESMODUR® N3300A. This mixture was then stirred briefly (until homogeneous), allowed to stand a few minutes to allow air bubbles to dissipate, and then a 1.0 g portion was poured gently, without stirring, into a loz. glass vial containing lOg of “Lily of the Valley” green fragrance oil (Wellington Fragrances). The matrix solution floated on top of the fragrance oil and the oil remained as a separate reservoir below it. The set time for the top (membrane) layer that gradually absorbed some of the fragrance oil, was 80 minutes. The vial was capped and allowed to cure for an additional 24 hours. After this time, the vial was suspended inverted. In this position, fragrance oil gradually permeated the membrane and evaporated, acting as a sustained release air freshener.

### Example 39

This example illustrates the preparation of a article containing an aromatic filler. To a glass mixing jar was charged 15 g of the Example 37 polyamine, 6 g of castor oil, and 9 g of commercial ground coffee and the mixture was stirred gently for 30 minutes at ambient temperature. To this viscous paste was then added 2.0 g of DEMODUR® N3300A. This mixture was then stirred briefly, allowed to stand a few minutes to allow any air bubbles to dissipate, poured (18.0 g used) into a disk-shaped flexible mold of uniform circumference of 8.25 inches, height of 0.25 inches, and top and bottom-width of 2.5 inches. The set time was recorded at 165 minutes. The mixture was covered and allowed to cure undisturbed for 24 hours. After this time the mold was stripped away from the object that was now fragrant (coffee odor), firm, flexible, and non-tacky to the touch.

### Example 40

This example illustrates the preparation of an article containing water. To a glass mixing jar was charged 20 g of the Example 37 polyamine, 20 g of “Snuggle Type” fragrance oil (from Alpha Aromatics), and 8 g of de-ionized water, and the mixture was stirred gently for 15 minutes at ambient temperature, resulting in a milky suspension of water in the matrix-fragrance solution. Blue dye (2 drops) was added to the mixture. To this light blue, milky mixture was then added 2.5 g of DESMODUR® N3300A. This mixture was then stirred briefly and allowed to stand a few minutes to allow any air bubbles to dissipate. Then a 31.0 g portion was poured into a bunt cake-shaped flexible silicone mold of uniform top-width of 1.75 inches, height of 0.75 inches, and bottom-width of 2.5 inches. The set time was recorded at 130 minutes. The mixture was covered and allowed to cure undisturbed for 24 hours. After this time the mold was stripped away from the cross-linked air freshener object that was now firm, milky, flexible, and non-tacky to the touch. The article gradually turned clear (starting from the edges and moving toward the center) as the water evaporated over a period of one month.

### Example 41

This example illustrates the preparation of a dispersion. Solution A: to a glass mixing jar was charged 8 g of the Example 37 polyamine and 8 g of FINSOLV® TN and the mixture was stirred gently for 15 minutes at ambient temperature. To this homogeneous mixture was then added 0.8 g of DESMODUR® N3300A. This mixture was then stirred briefly (until homogeneous), allowed to stand a few minutes to allow any air bubbles to dissipate. Solution B: to another glass mixing jar was charged 32 g deionized water and 0.8 g of surfactant (T-DET A-136). This mixture was stirred (10 minutes). Solution A was then poured into Solution B with stirring for 10 minutes. This blend of mixture A+B mixture was then poured into a metal pan and the water allowed to evaporate (24 hours). This yielded a white, lubricious powder, insoluble in toluene, of immobilized oil particles.

### Example 42

Representative of articles containing pesticide that can be prepared according to the present invention is the following controlled-release diethyl toluamide (DEET) device. A thorough mixture was made of DEET (20 parts), dimethyl adipate carrier (50 parts), benzaldehyde as fragrance and retardant (1.6 parts), the polyamide polyamine of Example 21 (26.8 parts) and a trace of orange dye. To this blend was then added with stirring DESMODUR® N3300 polyisocyanate (3.2 parts) and the final mixture poured into scallop-shaped silicone molds. After this cured, the scallop medallion so formed was a firm, non-tacky, and flexible solid.

### Example 43

Representative of articles containing pheromones that can be prepared according to the present invention is the following controlled-release device for the pheromone octadecanal. A thorough mixture was made of octadecanal (30 parts), FINSOLV® TN benzoate ester as carrier (30 parts), and the polyamide polyamine of Example 21 (35.5 parts). To this blend was then added with stirring DESMODUR® N3300 polyisocyanate (4.5 parts) and the final mixture poured into a cylindrical mold. After curing, the material formed was a firm, non-tacky, and flexible solid that could be sliced into small disks for use as lures.

### Example 44

This example illustrates the use of a styrene-maleic anhydride copolymer as the reactive partner with a polyamide polyamine for preparation of a lightly-scented disk-shaped air freshener. To a glass mixing vial was charged 6.0 g of a 25 wt % solution FINSOLV® TN solution of the Example 21 polyamide polyamine, 7.5 g of a 20 wt % solution of DYLARK® 232 poly(styrene-co-maleic anhydride, NOVA Chemicals), and ca. 2 g of “Ocean” fragrance oil (provided by Wellington, Inc.). The mixture was stirred gently for a few minutes at ambient temperature and blue dye (4 drops) added. The mixture was initially slightly turbid but cleared after a few more minutes and remained clear and apparently homogeneous. The mixture was then poured (about llg was used) into a disk-shaped polyethylene mold and allowed to stand undisturbed. The mixture set to a sticky, elastic mass inside about 2 hours and after 24 hours could be stripped from the mold. After this time the mold was stripped away from the cross-linked air freshener object that was now firm, transparent, and flexible with a light tack to the touch.

### Example 45

This example illustrates the use of a cationic surfactant to prepare an immobilized fragrance emulsion useful as a fabric softener. A blend of PAPA of Example 13 (4.0 g), “Cinnamon Chai” fragrance oil (3.0 g), and VARIQUAT® B1216 alkyl dimethyl benzyl ammonium chloride (80% active, Degussa Corporation, 1.0 g) was first prepared by warming and stirring the ingredients. To the blend was added water (9.0 g) and then, with stirring, DESMODUR® N3300A (0.65 g). The mixture soon became viscous and uniformly cloudy. It was storage stable and was dilutable with water, indicating it was an oil-in-water dispersion. Light scattering particle size measurement on the material determined the particle size distribution to be bi-modal, with about 50% of the weight of particles having a size grouping around 0.4 microns and the other 50% grouping around 3.0 microns.

### Example 46

This example illustrates the preparation of an immobilized cationic surfactant useful as a fabric softener. A blend of PAPA of Example 21 (3.0 g), DOWANOL® DPM (1.0 g) and VARIQUAT® B1216 alkyl dimethyl benzyl ammonium chloride (80% active, Degussa Corporation, 6.0 g) was first prepared by warming and stirring the ingredients and then cooling them to room temperature. A second blend was prepared of DOWANOL® DPM (4.2 g) and DESMODUR® N3300A (0.8 g). The two clear mixtures were then mixed together and immediately poured into a mold. The blended components set almost immediately and were firm enough to pick up out of the mold in less than 30 minutes. The final article contained 32% by weight active quaternary compound.

### Example 47

A secondary amine terminated polyamide polyamine (SATPP) was prepared by charging PRIPOL 1006 polymerized fatty acid (48.8 g, 219 meq acid) (Croda, Inc.; Edison, N.J.), JEFFAMINE D-2000 (54.9 g, 1000 meq amine) (Huntsman Corporation; The Woodlands, Tex.), and JEFFAMINE D-403 (4.88 g, 146 meq amine) (Huntsman Corporation; The Woodlands, Tex.) to a 250 mL glass flask equipped with a magnetic stir bar. The contents of the flask were heated to 100° C. with stirring under a stream of dry nitrogen and then 3-cyclohexylamine propylamine (CHAPA) (11.59 g, 78 meq amine) was added. The mixture was heated to 210-220° C. under a stream of dry nitrogen. After holding the mixture under these conditions for 4 hours, the reaction mixture was discharged to a container. The product was clear and viscous and had an acid number of 0.7, an amine number of 38.1, and a weight average molecular weight of 12,600 Daltons.

### Example 48

A secondary amine terminated polyamide polyamine (SATPP) was prepared by charging PRIPOL 1006 polymerized fatty acid (48.8 g, 219 meq acid) (Croda, Inc.; Edison, N.J.), JEFFAMINE D-2000 (54.9 g, 1000 meq amine) (Huntsman Corporation; The Woodlands, Tex.), and JEFFAMINE D-403 (4.88 g, 146 meq amine) (Huntsman Corporation; The Woodlands, Tex.) to a 250 mL glass flask equipped with a magnetic stir bar. The contents of the flask were heated to 210-220° C. with stirring under a stream of dry nitrogen and held at these conditions for 3 hours. The mixture was then cooled to 100° C. and aminoethylpiperazine (AEP) (9.5 g, 64.6 meq amine) was added. The resulting mixture was heated to 210-220° C. and held at these conditions for 4 hours. The reaction mixture was then discharged to a container to provide a clear and viscous product having an acid number of 1.5, an amine number of 32, and a weight average molecular weight of 36,700 Daltons.

### Examples 49-78

For each of examples 49-78 (see Table 1), a secondary amine terminated polyamide polyamine (SATPP) (1.25 g) and fragrance oils (3.5 g) were manually mixed in a glass vessel to obtain a homogenous solution. The fragrance oils were obtained from Belcan Inc. (Yonkers, N.Y.), Givaudan (Vernier, Switzerland), or Orlandi, Inc. (Farmingdale, N.Y.), as indicated in Table 6. The mixture was allowed to stand for 10 minutes. One equivalent of DESMODUR N 3300, an isocyanate hardener commercially available from Bayer Corporation (Pittsburgh, Pa.), was then added with manual stirring. The gel time was measured by observing the amount of time lapsed to provide a mixture no longer able to flow under its own weight. As shown in Table 1, each of the formulations using a SATPP immobilized the target fragrance oils within 2 minutes, implying that the SATPP amine functional groups did not interfere with the aldehyde functional groups in the fragrance oils.

TABLE 6

| | | | |
|---|---|---|---|
| | | | Set Time |
| Example | Fragrance Oil Type | SATPP | (minutes) |

Belcan Fragrances

| | | | |
|---|---|---|---|
| 49 | Lemon Sage | Example 48 | <2 |
| 50 | Lemon Sage | Example 47 | <2 |
| 51 | Lemon Sage | JEFFAMINE SD-2001 | <2 |
| 52 | Juicy Apple | Example 48 | <2 |
| 53 | Juicy Apple | Example 47 | <2 |
| 54 | Juicy Apple | JEFFAMINE SD-2001 | <2 |
| 55 | Applewood | Example 48 | <2 |
| 56 | Applewood | Example 47 | <2 |
| 57 | Applewood | JEFFAMINE SD-2001 | <2 |
| 58 | Strawberry | Example 48 | <2 |
| 59 | Strawberry | Example 47 | <2 |
| 60 | Strawberry | JEFFAMINE SD-2001 | <2 |
| 61 | Honeysuckle | Example 48 | <2 |
| 62 | Honeysuckle | Example 47 | <2 |
| 63 | Honeysuckle | JEFFAMINE SD-2001 | <2 |
| 64 | Apricot Mango | Example 48 | <2 |
| 65 | Apricot Mango | Example 47 | <2 |
| 66 | Apricot Mango | JEFFAMINE SD-2001 | <2 |
| 67 | Fresh Rain | Example 48 | <2 |
| 68 | Fresh Rain | Example 47 | <2 |
| 69 | Fresh Rain | JEFFAMINE SD-2001 | <2 |

Givaudan Fragrances

| | | | |
|---|---|---|---|
| 70 | Natalie | Example 48 | <2 |
| 71 | Natalie | Example 47 | <2 |
| 72 | Natalie | JEFFAMINE SD-2001 | <2 |

Orlandi Fragrance

| | | | |
|---|---|---|---|
| 73 | Garden Sage | Example 48 | <2 |
| 74 | Garden Sage | Example 47 | <2 |
| 75 | Garden Sage | JEFFAMINE SD-2001 | <2 |
| 76 | Lime Basil | Example 48 | <2 |
| 77 | Lime Basil | Example 47 | <2 |
| 78 | Lime Basil | JEFFAMINE SD-2001 | <2 |

### Examples 79-90

Examples 79-90 illustrate the gel setting time differences between secondary amine terminated polyamide polyamine (SATPP) and primary amine terminated polyamide polyamine (PATPP), as shown in Table 7. The formulations using PATPP showed longer and varying set times for different fragrance oils. On the contrary, all the formulations using SATPP had set times within 2 minutes. The immobilized and intermixed fragrance oils were prepared according to the procedure for Examples 49-78.

TABLE 7

| | Fragrance | Hardener | | SATPP | Set Time |
|---|---|---|---|---|---|
| | Oil Type | Bayer | Polyamine | PATPP | (minutes) |
| Ex. 79 | Cherry | N3300 | Ex. 48 | SATPP | <2 |
| Ex. 80 | Cherry | N3300 | JEFFAMINE SD-2001 | SATPP | <2 |
| Ex. 81 | Cherry | N3300 | JEFFAMINE D-2000 | PATPP | Never Set |
| Ex. 82 | Cherry | N3300 | SYLVACLEAR IM 700 | PATPP | Never Set |
| Ex. 83 | Orange | N3300 | Ex. 48 | SATPP | <2 |
| Ex. 84 | Orange | N3300 | JEFFAMINE SD-2001 | SATPP | <2 |
| Ex. 85 | Orange | N3300 | JEFFAMINE D-2000 | PATPP | 30 |
| Ex. 86 | Orange | N3300 | SYLVACLEAR IM 700 | PATPP | 45 |
| Ex. 87 | Berry | N3300 | Ex. 48 | SATPP | <2 |
| Ex. 88 | Berry | N3300 | JEFFAMINE SD-2001 | SATPP | <2 |
| Ex. 89 | Berry | N3300 | JEFFAMINE D-2000 | PATPP | <2 |
| Ex. 90 | Berry | N3300 | SYLVACLEAR IM 700 | PATPP | <2 |

### Examples 91-94

For each of examples 91-94, a secondary amine terminated polyamide polyamine (SATPP), SYLVACLEAR IM 800, a polyamide polyamine terminated with a carbonyl-substituted aromatic amine commercially available from Arizona Chemical Company (Jacksonville, Fla.), and fragrance oils were manually mixed in a glass vessel to obtain a homogenous solution. The mixture was allowed to stand for 10 minutes. One equivalent of isocyanate hardener DESMODUR N 3300 (Bayer Corporation; Pittsburgh, Pa.) was then added with manual stirring. The gel time was measured by observing the amount of time lapsed to provide a mixture no longer able to flow under its own weight. Table 8 illustrates the gel setting time for the formulations with SATPP can be adjusted by blending with polyamide polyamines terminated with carbonyl-substituted aromatic amine.

TABLE 8

| Components | Ex. 91 | Ex. 92 | Ex. 93 | Ex. 94 |
|---|---|---|---|---|
| Ex. 48 | 0.75 | 0 | 0.43 | 0 |
| JEFFAMINE SD-2001 | 0 | 0.71 | 0.0 | 0.41 |
| SYLVACLEAR IM 800 | 0.68 | 0.69 | 0.9 | 0.96 |
| Orange fragrance | 3.6 | 3.6 | 3.6 | 3.6 |
| DESMODUR N3300 | 0.31 | 0.3 | 0.29 | 0.32 |
| Set Time, minutes | 310 | 198 | 355 | 243 |

### Examples 95-104

For each of examples 95-104, immobilized fragrance oil dispersions were prepared according to the following generic process. Details on the individual components for each of the dispersions are described in Tables 9 and 10. To form Part A, the indicated amounts of water, 1% METHOCEL 311 cellulose water solution (Dow Chemical; Midland, Mich.), FINSOLV-TN (an alkyl benzoate commercially available from Innospec Active Chemicals (Edison, N.J.), and a surfactant ARQUAD 18-50 (Akzo Nobel Surface Chemistry LLC; Chicago, Ill.) were charged into a 100 mL plastic cup equipped with an impeller. Part B was prepared by pre-mixing the indicated amounts of fragrance Natalie (Givaudan; Vernier, Switzerland) and polyamide polyamine SYLVACLEAR IM 700 (Arizona Chemical Company; Jacksonville, Fla.) to form a homogenous solution in 2 minutes. Part B was dispersed dropwise into Part A aqueous phase immediately at the indicated rpm. Part B addition was completed in 3 minutes. After addition, the mixture was agitated at the indicated rpm for 60 minutes and discharged to a container. The dispersions were milky solutions. For some batches, the indicated amount of additional surfactant ARQUAD 18-50 was added and mixed for 5 minutes.

TABLE 9

| | Ex. 95 | Ex. 96 | Ex. 97 | Ex. 98 | Ex. 99 |
|---|---|---|---|---|---|
| Mixing speed (rpm) | 600 | 600 | 700 | 700 | 700 |
| SYLVACLEAR IM700 | 2.25 | 2.25 | 2.25 | 2.25 | 2.25 |
| (grams) | | | | | |
| Natalie (fragrance) | 5 | 5 | 5 | 5 | 5 |
| (grams) | | | | | |
| FINSOLV-TN (grams) | 0 | 0 | 0 | 0 | 0 |
| 1% METHOCEL 311 | 6 | 6 | 6 | 6 | 6 |
| (grams) | | | | | |
| Water (grams) | 6 | 6 | 6 | 6 | 6 |
| ARQUAD 18-50 (first | 0.7 | 0.7 | 0.7 | 0.7 | 1.7 |
| addition) (grams) | | | | | |
| DESMODUR N3300 | 0.35 | 0.35 | 0.35 | 0.35 | 0.35 |
| (hardener) (grams) | | | | | |
| ARQUAD 18-50 (second | 0 | 1 | 0 | 1 | 0 |
| addition) (grams) | | | | | |
| Total weight (grams) | 20.3 | 21.3 | 20.3 | 21.3 | 21.3 |
| Mean particle size | 69.4 | 58.02 | 45.2 | 45.4 | 25.9 |
| (microns) | | | | | |

TABLE 10

| | Ex. 100 | Ex. 101 | Ex. 102 | Ex. 103 | Ex. 104 |
|---|---|---|---|---|---|
| Mixing speed (rpm) | 700 | 800 | 1000 | 1000 | 1000 |
| SYLVACLEAR IM700 | 2.25 | 2.25 | 2.25 | 2.25 | 2.25 |
| (grams) | | | | | |
| Natalie (fragrance) | 5 | 5 | 5 | 5 | 5 |
| (grams) | | | | | |
| FINSOLV-TN (grams) | 0 | 0 | 0.8 | 0 | 0.8 |
| 1% METHOCEL 311 | 6 | 6 | 4.5 | 6 | 4.5 |
| Cellulose (grams) | | | | | |
| Water (grams) | 6 | 6 | 6 | 6 | 6 |
| ARQUAD 18-50 (first | 2.3 | 1.7 | 1.7 | 1.7 | 1.7 |
| addition) (grams) | | | | | |
| DESMODUR N3300 | 0.35 | 0.35 | 0.35 | 0.35 | 0.35 |
| (hardener) | | | | | |
| ARQUAD 18-50 (second | 0 | 0 | 0 | 0 | 0 |
| addition) (grams) | | | | | |
| Total weight (grams) | 21.9 | 21.3 | 20.6 | 21.3 | 20.6 |
| Mean particle size | 38.6 | 31.8 | 21.52 | 25.91 | 12.82 |
| (microns) | | | | | |

### Example 105-107

For each of Examples 105-107, the fragrance oil dispersions were prepared according to the following generic process, with details regarding the individual components shown in Table 11. The indicated amounts of SYLVACLEAR IM700 (Arizona Chemical Company; Jacksonville, Fla.) and the fragrance Berry (Belmay Fragrances Ltd.; Yonkers, N.Y.) were pre-mixed to form a homogenous solution. The solution was added dropwise to an aqueous solution having the indicated amounts of water, 1% METHOCEL 311 (Dow Chemical; Midland, Mich.) water solution, and surfactant ARQUAD 18-50 (Akzo Nobel Surface Chemistry LLC; Chicago, Ill.) with mixing at the indicated rpm in a 100 mL plastic cup. The addition was completed within 3 minutes and the mixing was continued for 30 minutes. The hardener DESMODUR N3300 (Bayer Corporation; Pittsburgh, Pa.) was added dropwise, mixing was allowed to continue for 60 minutes, and the resulting dispersion was discharged to a container. The dispersions were milky solutions.

TABLE 11

| | Ex. 105 | Ex. 106 | Ex. 107 |
|---|---|---|---|
| Agitation speed (rpm) | 1500 | 700 | 700 |
| SYLVACLEAR IM700 | 2.34 | 2.24 | 2.24 |
| (grams) | | | |
| Berry fragrance | 7.22 | 7.2 | 7.2 |
| (grams) | | | |
| 1% METHOCEL 311 | 5.7 | 8 | 8 |
| Cellulose (grams) | | | |
| Water (grams) | 5.7 | 5 | 5 |
| ARQUAD 18-50 (grams) | 0.66 | 0.69 | 1.37 |
| DESMODUR N3300 | 0.39 | 0.41 | 0.41 |
| (hardener) (grams) | | | |
| Total weight (grams) | 22.01 | 23.54 | 24.22 |
| Mean particle size | 4.29 | 22.37 | 5.67 |
| (microns) | | | |

### Example 108

SYLVACLEAR IM800 (4.50 g) (Arizona Chemical Company; Jacksonville, Ill.) and 10.50 g of the fragrance Natalie (Givaudan; Vernier, Switzerland) were mixed to obtain a homogenous solution. Then 0.58 g DESMODUR N3300 (Bayer Corporation; Pittsburgh, Pa.) was added to the solution and mixed well to form a homogenous solution in 5 minutes. The solution was added dropwise to an aqueous solution having 12.50 g water, 5.0 g 1% METHOCEL 311 water solution (Dow Chemical; Midland, Mich.), and 2.68 g surfactant ARQUAD 16-50 (Akzo Nobel Surface Chemistry LLC; Chicago, Ill.) with mixing at 1200 rpm in a 100 mL plastic cup. The addition was completed within 3 minutes. The mixing was continued for 130 minutes and the resulting mixture was discharged to a container. The dispersions were milky solutions. The mean particle size was 10.5 microns.

### Example 109

SYLVACLEAR IM800 (4.50 g) (Arizona Chemical Company), 5.5 g of solvent FINSOLV-TN (a solvent commercially available from Innospec Active Chemicals (Edison, N.J.)), and 5.0 g N,N-diethyl-meta-toluamide (DEET) were mixed to obtain a homogenous solution. Then 0.58 g DESMODUR N3300 (Bayer Corporation; Pittsburgh, Pa.) was added to the solution and mixed well to form a homogenous solution. The solution was added dropwise to an aqueous solution having 12.50 g water, 5.0 g 1% METHOCEL 311 water solution (Dow Chemical; Midland, Mich.), and 2.68 g ARQUAD16-50 (Akzo Nobel Surface Chemistry LLC; Chicago, Ill.) with mixing at 1200 rpm in a 100 mL plastic cup. The addition was completed within 3 minutes. The mixing was continued for 120 minutes and the resulting mixture was discharged to a container. The dispersions were milky solutions.

### Example 110

SYLVACLEAR IM800 (4.50 g) (Arizona Chemical Company; Jacksonville, Fla.), 5.5 g of FINSOLV-TN (a solvent commercially available from Innospec Active Chemicals (Edison, N.J.)), and 5.0 g sumithrin were mixed to obtain a homogenous solution. Then 0.58 g DESMODUR N3300 (Bayer Corporation; Pittsburgh, Pa.) was added to the solution and mixed well to form a homogenous solution. The solution was added dropwise to an aqueous solution having 12.50 g water, 5.0 g 1% METHOCEL 311 water solution (Dow Chemical; Midland, Mich.), and 2.68 g surfactant ARQUAD 16-50 (Akzo Nobel Surface Chemistry LLC; Chicago, Ill.) with mixing at 1200 rpm in a 100 mL plastic cup. The addition was completed within 3 minutes. The mixing was continued for 120 minutes and discharged to a container. The dispersions were milky solutions.

The compositions, methods, and apparatuses of the appended claims are not limited in scope by the specific compositions, methods, and articles described herein, which are intended as illustrations of a few aspects of the claims and any compositions, methods, and articles that are functionally equivalent are intended to fall within the scope of the claims. Various modifications of the compositions, methods, and articles in addition to those shown and described herein are intended to fall within the scope of the appended claims. Further, while only certain representative composition materials and method steps disclosed herein are specifically described, other combinations of the composition materials and method steps also are intended to fall within the scope of the appended claims, even if not specifically recited. Thus, a combination of steps, elements, components, or constituents can be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of and “consisting of can be used in place of “comprising” and “including” to provide for more specific embodiments and are also disclosed.

What is claimed is:

1. A composition, comprising:
a polymeric matrix comprising the reaction product of a polyamine and a compound having at least two functional groups, the functional groups selected from the group consisting of epoxy groups, isocyanate groups, anhydride groups, and acrylate groups; and
an active liquid intermixed with at least a portion of the polymeric matrix,
wherein the polyamine and the compound are reacted in the presence of the active liquid.

2. The composition of claim 1, wherein the polyamine is a polyamide polyamine.

3. The composition of claim 2, wherein the polyamine is a secondary amine terminated polyamine.

4. The composition of claim 1, wherein the polyamine is a secondary amine terminated polyamine.

5. The composition of claim 1, wherein the polyamine is a non-water soluble polyamide polyamine with a molecular weight in the range of 4,000 to 30,000 Daltons.

6. The composition of claim 1, wherein the reactive amine groups of the polyamine include amino groups derived from at least one of ortho-aminobenzoic acid or para-aminobenzoic acid.

7. The composition of claim 1, wherein the compound is a compound having at least two isocyanate functional groups.

8. The composition of claim 1, wherein the active liquid is present in an amount from 10 weight % to 85 weight % based on the weight of the composition.

9. The composition of claim 1, wherein the active liquid is present in an amount from 50 weight % to 85 weight % based on the weight of the composition.

10. The composition of claim 1, wherein the active liquid includes a therapeutic active liquid, a nutraceutical active liquid, a cosmeceutical active liquid, a pesticidal active liquid, a laundry care active liquid, a fragrance, or a mixture thereof

11. The composition of claim 1, wherein the compound includes at least one non-aromatic isocyanate compound.

12. The composition of claim 1, wherein the composition is in the form of a gel.

13. The composition of claim 1, wherein the composition is in the form of a particle and is present in an aqueous dispersion.

14. The composition of claim 13, wherein the particle size of the particle is from 1 micron to 100 microns.

15. The composition of claim 13, wherein the particle size of the particle is from 2 microns to 15 microns.

16. An article comprising a porous support material and the composition of claim 1.

17. A method of preparing a composition, comprising:
reacting a polyamine with a compound having at least two functional groups, the functional groups selected from the group consisting of epoxy groups, isocyanate groups, anhydride groups, and acrylate groups in the presence of an active liquid.

18. The method of claim 17, wherein the polyamine is liquid at room temperature.

19. The method of claim 17, wherein the polyamine has an amine number of from 10 to 100 meq KOH/g.

20. The method of claim 17, wherein the polyamine has a viscosity of 500 cP or less at 150° C.

21. The method of claim 17, wherein the reacting step occurs at room temperature.
